SEQUENCE LISTING (29 sequences)

SEQ ID NO: 1 - protein, 243 aa, Aspergillus oryzae
  SIGNAL 1..22
  PROPEP 23..37
  PEPTIDE 38..243: note = Mature polypeptide
MQLTKSLLVFALYMFGTQHVLAVPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 2 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 3 - protein, 204 aa, Aspergillus oryzae
  PEPTIDE 1..204: note = Mature polypeptide
KTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 4 - protein, 182 aa, Bacillus cibi [sequence omitted]

SEQ ID NO: 5 - protein, 275 aa, Bacillus amyloliquefaciens [sequence omitted]

SEQ ID NO: 6 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 7 - protein, 269 aa, Synthetic construct
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 8 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 9 - protein, 274 aa, Synthetic construct
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSSGSGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 10 - protein, 324 aa, Terribacillus saccharophilus [sequence omitted]

SEQ ID NO: 11 - protein, 331 aa, Lactobacillus paraplantarum
NSSTLNTSQGVMLDLGRHPLDETAIKAVISAAAEQHMQYVELHLSDNEHLCFQSAYLGNAASATVLSATTLEQLVAYANQLNIELVPDVDLPSHAGAILRQLQQTHPDIYNTVKLDDETIDYTKPAAISLATTLYGELDASFNNQSQHDLMLGADEVPGSASAYIELTTFINQVSRFQNQHGFNTSIWNDSLLKNELTRLDSNITINYWSQSGNNTDVAIIADRYANRVSVPDILASGHPIVNCNSYATYYQIKNIGNVNDDDYFINYLNHTFRPNIFNEIDTNGHNQDWTIEDGVTTNGILVSLWGADSEHVTPTAIVNFIKRMTIPRSF

SEQ ID NO: 12-15 - skipped (4 entries)

SEQ ID NO: 16 - protein, 4 aa, Synthetic construct
QSTL

SEQ ID NO: 17 - protein, 5 aa, Synthetic construct
NKFFY

SEQ ID NO: 18 - protein, 5 aa, Synthetic construct
  VARIANT 4: note = Xaa = D (Asp) or R (Arg)
NLDXS

SEQ ID NO: 19 - protein, 324 aa, Terribacillus saccharophilus
KDQEKGITIDISRKYYSIGTLKAIVDEINANGGDYLQLHFSDNESYAIASEFLGQNSENPNSTYLTKKELLSLIAYSNDRNIMVIPDIDLPAHSKGWLNVMKEKDSGLYTDIVTDYSEDTLDYHNNAAALYTANQLLDEVLDLFYQPKFAGKQRIVLGGDEVPGSGAHQTDFIRFMNQIDETAKASNYEPQMWNDSITPEGIQNLDRSFSILYWKQSTLSSGAQGLDVQNFEEKGFSVYNYNAYSLYFLPSTRFTQEDITEQIDYMKWAYAYNKFFYISDYYKQVDTSNVKGSSLVFWGEHANDLSQEGLLEQEKPLIQNFLSL

SEQ ID NO: 20 - protein, 323 aa, Staphylococcus cohnii
QDFQKGINVDIARKDYSLKSLKKIVDTIHENNGDYLQLHFSDNENYAIESQFFKHENIASQNYLSQQELKNLIHYSNKLNIMVVPEFDLPSHSKAWLLLLKNENSNLHENIVSDYSDETIDFFSNQKALEISKRQIKEILNLFHQPNFQKEQRIVLGGDEVPGGKSYQNDFINFMNEIGEYAYQNGYEPQIWNDSITKNGLKLLKNYFSVIFWKQSNNENNEPGITVEDFLDYNFKVYNYNFYSLYFLPSKNYSPTDIEEQTSYISWAYNHNSFYYLKNPYYEVDSLNIQGSALSFWGEHATGMREEEVLNQELPLIRTYLNK

SEQ ID NO: 21 - protein, 321 aa, Staphylococcus fleurettii
ESIQEGVSVDIARKEYSLESLKQIVDTIHENNGQYLQLHFSDDENYAIESDYFSHQGIPNENYLTKAEIKSLIAYSNELNVMVVPDIDFPSHSKALLSLIKNEDKDLYNQIISDYSDNTFDFFSNDKALAISKRHIGEITTLFNQPKYNGQQRIVLGGDEVPGGGAYQSDFISYMNNIGSYAAGQGYEPQMWNDMISHEGIKSLNDTFSILYWKQNENSKSDLTVEDFAEYDFKIYNYNFYSLYFLPSNQFTNADIEEQADYISWAYAYNKFFYTNEPYQEVDSDNVKGSALSFWGEDALNMSQTELINQEIPLIKAYFSS

SEQ ID NO: 22 - protein, 359 aa, Aggregatibacter actinomycetemcomitans
CVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 23-26 - skipped (4 entries)

SEQ ID NO: 27 - protein, 7 aa, Synthetic construct
  VARIANT 1: note = Xaa = Asp (D) or Met (M) or Leu (L)
  VARIANT 2: note = Xaa = Ser (S) or Thr (T)
  VARIANT 7: note = Xaa = Asp (D) or Asn (N)
XXGYSRX

SEQ ID NO: 28 - protein, 8 aa, Synthetic construct
  VARIANT 3: note = Xaa = any amino acid
ASXNRSKG

SEQ ID NO: 29 - protein, 269 aa, Synthetic construct
AQTVPWGISRVQAPAAHNRGLTGSGVKVAVLDTGISTHPDLRIRGGASFVPGEPSTQDGNGHGTHVAGTIAALDNSIGVLGVAPNAELYAVKVLGASGSGSVSSIAQGLEWAGNNMMHVANLSLGQPSPSATLEQAVNSATSRGVLVVAASGNSGAGSISYPARYANAMAVGATDQNNERASFSQYGAGLDIVAPGVNILSTWPGSTYASLNGTSMATPHVAGAAALVKQKNPSWSNVQIRNHLKNTATSLGDTWEYGSGLVNAEAATR

(19) United States
(12) Patent Application Publication
(10) Pub. No.: US 2025/0295120 A1
(43) Pub. Date: Sep. 25, 2025

(54) CLEANING METHOD, USE OF ENZYMES AND CLEANING COMPOSITION

(71) Applicant: Novozymes A/S, Bagsvaerd (DK)

(72) Inventors: Yuan XU, Beijing (CN); Inge Byg ULBRINK, Bagsvaerd (DK); Rebecca Munk VEJBORG, Allerod (DK); Rasmus Rune HANSEN, Ballerup (DK); Tianhu ZHAO, Beijing (CN); Yang LIU, Beijing (CN); Wei WEI, Tianjin (CN); Xiaoxi ZHANG, Tianjin (CN)

(73) Assignee: Novozymes A/S, Bagsvaerd (DK)

(21) Appl. No.: 18/728,035

(22) PCT Filed: Jan. 16, 2023
(86) PCT No.: PCT/CN2023/072378

§ 371 Date: Jul. 10, 2024

(30) Foreign Application Priority Data
Jan. 21, 2022 (WO) PCT/CN2022/073148

(51) Int. Cl. A01N 63/50; A01P 1/00

(52) U.S. Cl. CPC A01N 63/50; A01P 1/00

(57) ABSTRACT

Provided is a method for cleaning a medical device comprising contacting the device with a wash liquor comprising two or more enzymes selected from the group consisting of a protease, an enzyme having DNase activity and an enzyme having hexosaminidase activity.

### REFERENCE TO A SEQUENCE LISTING

This application contains a Sequence Listing in computer readable form, which is incorporated herein by reference.

### FIELD OF THE INVENTION

The present invention concerns a cleaning method for a medical device, and use of an enzyme composition comprising two or more enzymes selected from the group consisting of a protease, an enzyme having DNase activity and an enzyme having hexosaminidase activity for cleaning a medical device. The invention further relates to a cleaning composition comprising said enzyme composition.

### BACKGROUND OF INVENTION

Medical devices are often heavily contaminated with organic soil as a result of their use. Before re-use, it is essential that the devices are properly cleaned and disinfected.

The nature and extent of contamination in the healthcare environment is far greater than that normally found in a domestic environment, and the need for efficient cleaning is high. Of particular concern are microorganisms which are resistant to multiple types of antibiotics as these are over-represented in healthcare environments. Accordingly, use of insufficiently reprocessed medical equipment poses a high risk of cross-infection to other patients, whose immune systems may already be compromised by illness, injury or the trauma of invasive medical procedures.

Typically, the procedure for reprocessing of medical devices comprises washing the device to remove organic materials, rinsing, disinfection and drying. Cleaned medical devices are often still soiled with organic material, which can significantly decrease the efficacy of the subsequent disinfection procedure.

WO 2017/129331 discloses a method for cleaning of medical and dental instruments using a protease.

WO 2019/086532 discloses a method of cleaning a medical device using a hexosaminidase having beta-N-acetylglucosaminidase activity.

There remains a need for improved compositions and methods for cleaning medical devices that can more effectively remove organic soil.

### SUMMARY OF THE INVENTION

The present invention concerns a method of cleaning a medical device comprising the steps of:
(a) contacting the medical device with a wash liquor comprising two or more enzymes selected from the group consisting of a protease, an enzyme having DNase activity and an enzyme having hexosaminidase activity; and optionally
(b) rinsing the medical device.

The present invention further concerns use of an enzyme composition for cleaning a medical device, wherein the enzyme composition comprises two or more enzymes selected from the group consisting of a protease, an enzyme having DNase activity and an enzyme having hexosaminidase activity.

In addition, the present invention concerns a composition for cleaning a medical device, comprising a surfactant and two or more enzymes selected from the group consisting of a protease, an enzyme having DNase activity and an enzyme having hexosaminidase activity.

### Definitions

As used herein, the singular forms “a”, “an”, and “the” are intended to include the plural forms as well, unless the context clearly indicates otherwise.

DNase: The term “DNase” means a polypeptide/enzyme with DNase activity that catalyzes the hydrolytic cleavage of phosphodiester linkages in the DNA backbone, thus degrading DNA. Exo-deoxyribonucleases cut or cleave residues at the end of the DNA backbone, whereas endo-deoxyribonucleases cleave or cut within the DNA backbone. A DNase may cleave only double-stranded DNA or may cleave double stranded and single stranded DNA. The term “DNases” and the expression “a polypeptide with/having DNase activity” or the expression “an enzyme with/having DNase activity” are used interchangeably throughout the application.

For purposes of the present invention, DNase activity is determined according to the procedure described in the Assay I. In one embodiment of the present invention, the DNases have at least 50%, e.g., at least 60%, at least 70%, at least 80%, at least 90%, at least 100%, at least 110%, at least 120%, or at least 150% of the DNase activity of the mature polypeptide of SEQ ID NO: 1, an enzyme comprising or consisting of the sequence set forth in SEQ ID NO: 2, an enzyme comprising or consisting of the sequence set forth in SEQ ID NO: 3, or an enzyme comprising or consisting of the mature polypeptide of SEQ ID NO: 4.

Hexosaminidase: The term “hexosaminidase” means a polypeptide having hexosaminidase activity (hexosaminidases), and includes EC 3.2.1., e.g. enzymes that catalyze the hydrolysis of N-acetyl-D-hexosamine or N-acetyl-glucosamine polymers found e.g. in biofilm. The term includes dispersins and includes polypeptides having N-acetylglucosaminidase activity and β-N-acetylglucosamininidase activity. The term “polypeptide having hexosaminidase activity” may be used interchangeably with the term hexosaminidase and similarly the term “polypeptide having β-N-acetylglucosaminidase activity” may be used interchangeably with the term β-N-acetylglucosamininidase. For purposes of the present invention, hexosaminidase activity is determined according to the procedure described in Assay II. In one aspect, the hexosaminidases of the present invention have at least 40%, e.g., at least 50%, at least 60%, at least 70%, at least 80%, at least 90%, at least 100%, at least 110%, or at least 120% of the hexosaminidase activity of the mature polypeptide of SEQ ID NO: 10.

Dispersin: The term “dispersin” and the abbreviation “Dsp” means a polypeptide having hexosaminidase activity, EC 3.2.1.-, that catalyzes the hydrolysis of β-1,6-glycosidic linkages of N-acetyl-glucosamine polymers (poly-N-acetylglucosamine) found e.g. in biofilm.

Bacterial: The term “bacterial” in relation to a polypeptide such as an enzyme refers to both polypeptides encoded by and thus directly derivable from the genome of a bacteria as well as genetically modified variants thereof, for example variants of a bacterial enzyme that have been modified using protein engineering techniques to result in an enzyme with desired characteristics such as improved stability and/or increased enzymatic activity. A bacterial polypeptide that is directly derived from a bacterium may be referred to a wildtype enzyme. A variant of a wildtype bacterial enzyme may be referred to as being “substantially homologous” to the wildtype sequence, which denotes an enzyme having at least 80% sequence identity, such as at least 85%, at least 90%, at least 95%, or at least 96%, 97%, 98% or 99% sequence identity to the amino acid sequence of a given wildtype enzyme.

Fungal: The term “fungal” in relation to a polypeptide such as an enzyme refers to both polypeptides encoded by and thus directly derivable from the genome of a fungus as well as genetically modified variants thereof, for example variants of a fungal enzyme that have been modified using protein engineering techniques to result in an enzyme with desired characteristics such as improved stability and/or increased enzymatic activity. A fungal polypeptide that is directly derived from a fungus may be referred to a wildtype enzyme. A variant of a wildtype fungal enzyme may be referred to as being “substantially homologous” to the wildtype sequence, which denotes an enzyme having at least 80% sequence identity, such as at least 85%, at least 90%, at least 95%, or at least 96%, 97%, 98% or 99% sequence identity to the amino acid sequence of a given wildtype enzyme.

Biofilm: A biofilm may be produced by any group of microorganisms in which cells stick to each other or stick to a surface, which in the context of the present invention is in particular the surface of a medical device. These adherent cells are frequently embedded within a self-produced matrix of extracellular polymeric substance (EPS). Biofilm EPS is a polymeric conglomeration generally composed of extracellular DNA, proteins, and polysaccharides. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community.

On medical devices biofilm producing bacteria can be e.g. found among the following species: Escherichia coli, Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538, Streptococcus (e.g., S. pyogenes, S. agalacticae or S. pneumoniae), Haemophilus influenzae, Pseudomonas aeruginosa, Clostridium perfringens, Chlamydia trachomatis, Candida albicans, Bacillus anthracis.

A wide range of bacterial and fungal microorganisms have been found to produce poly-N-acetylglucosamine (PNAG) or PNAG-like surface polysaccharides, including but not limited to Bacillus subtillis, Escherichia coli, Staphylococcus aureus, Staphylococcus epidermidis, Pseudomonas fluorescens, Yersinia pestis, Aggregatibacter actinomycetemcomitans, Streptococcus pyogenes, Streptococcus dysgalactiae (group C strep), Enterococcus faecalis, Listeria monocytogenes, Clostridium difficile, Mycobacterium tuberculosis, Mycobacterium smegmatis; Neisseria meningitides, Neisseria gonorrhea, nontypable Haemophilus influenzae, Haemophilus ducreyi, Helicobacter pylori, Campylobacter jejuni; Citrobacter rodentium, Salmonella enterica serovars typhi, Salmonella typhimurium, Candida albicans, Aspergillus flavus, Fusarium solani, and Cryptococcus neoformans.

Extracellular DNA (eDNA) is a common matrix component in microbial biofilms and has been identified in species including, but not limited to, Acinetobacter baumannii, Actinobacillus actinomycetemcomitans, Bdellovibrio bacterivorous, Bordetella pertussis, Bordetella bronchiseptica, Campylobacter jejuni, Comamonas denitrificans, Escherichia coli, Haemophilus influenza, Klebsiella pneumoniae, Neisseria meningitides, Pseudomonas aeruginosa, Shewanella oneidensis, Vibrio cholera, Gram-positive bacteria, Bacillus licheniformis, Bacillus subtilis, Enterococcus faecalis, Listeria monocytogenes, Micrococcus luteus, Staphylococcus aureus, Staphylococcus epidermidis, Staphylococcus haemolyticus, Streptococcus anginosus, Streptococcus constellatus, Streptococcus salivarius, Staphylococcus lugdunesis, Streptococcus intermedius, Streptococcus intermedius, Streptococcus mutans, Streptococcus pneumoniae, Streptococcus pyogenes, Aspergillus fumigatus and Candida albicans.

Most biofilms comprise biofilm or EPS from bacteria of many different species and are thus “poly-cultural”.

Clade: a group of polypeptides clustered together based on homologous features traced to a common ancestor. Polypeptide clades can be visualized as phylogenetic trees, and a clade is a group of polypeptides that consists of a common ancestor and all its lineal descendants, e.g. the Terribacillus clade or clade of Terribacillus is a group of enzymes all related to the same ancestor and sharing common properties.

Cleaning components: A “cleaning component” is an ingredient which is different from the enzymes according to this invention and is defined herein to mean the types of chemicals which can be used in cleaning compositions. Examples of cleaning components are alkalis, surfactants, hydrotropes, builders, co-builders, chelators or chelating agents, bleaching system or bleach components, polymers, suds suppressors, dispersants, bactericides, fungicides, corrosion inhibitors, soil suspending agents, soil release polymers, anti-redeposition agents, enzyme inhibitors or stabilizers, enzyme activators, antioxidants, preservatives and solubilizers.

Cleaning composition: The term “cleaning composition (may also be referred to as “detergent composition”) refers to compositions that find use in the removal of undesired compounds from items to be cleaned, such as a medical device. The cleaning compositions of the present invention are in particular adapted to medical cleaning. The term encompasses any materials/compounds selected for the particular type of cleaning composition desired and the form of the product (e.g., liquid, gel, powder, granulate, foam, or spray compositions). In addition to containing the enzymes of the invention, the cleaning composition of the present invention may contain one or more additional enzymes (such as amylases, lipases, cellulases, mannanases, hemicellulases, peroxidases, xylanases, phospholipases, esterases, cutinases, keratinases, reductases, oxidases, phenoloxidases, lipoxygenases, ligninases, pullulanases, malanases, β-glucanases, arabinosidases, hyaluronidases, laccases, perhydrolases and peroxidases, or any mixture thereof), and/or cleaning components as described above.

Deep cleaning: By the term “deep cleaning” is meant reduction or removal of components of biofilm, such as EPS or parts hereof, polysaccharides, PNAG (poly-N-acetylglucosamine), proteins, DNA, soil or other components present in the biofilm.

Enzyme detergency benefit: The term “enzyme detergency benefit” is defined herein as the advantageous effect an enzyme may add to a cleaning composition compared to the same composition without the enzyme. Important detergency benefits which can be provided by enzymes are stain removal with no or very little visible soils after washing and/or cleaning, or prevention or reduction of redeposition of soils released in the washing process (an effect that also is termed anti-redeposition). In the context of the present invention, this may also include removal of “invisible” soils that may otherwise remain on medical devices after cleaning with a composition that does not contain the enzymes of the invention. For the purpose of the present invention, the enzyme detergency benefit may be evaluated by biofilm reduction benefit as described in Example 1.

Mature polypeptide: The term “mature polypeptide” means a polypeptide in its final form following translation and any post-translational modifications, such as N-terminal processing, C-terminal truncation, glycosylation, phosphorylation, etc.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labeled “longest identity” is calculated as follows:

(Identical Residues×100)/(Length of Alignment−Total Number of Gaps in Alignment)

Variant: The term “variant” means a polypeptide/enzyme having the same type of activity as the parent enzyme and comprising an alteration, i.e., a substitution, insertion, and/or deletion, at one or more positions compared to the amino acid sequence of the parent. A substitution means replacement of the amino acid occupying a position with a different amino acid; a deletion means removal of the amino acid occupying a position; and an insertion means adding an amino acid adjacent to and immediately following the amino acid occupying a position.

In the context of the present invention, a variant may e.g. be a variant of an identified DNase that has the enzymatic activity of the parent, i.e. the capacity of catalyzing the hydrolytic cleavage of phosphodiester linkages in the DNA backbone (deoxyribonuclease activity). In one embodiment, the deoxyribonuclease activity of the variant is increased with reference to the parent DNase, e.g. the polypeptide of SEQ ID NO: 2 or 4.

In the context of the present invention, a variant may also e.g. be a variant of an identified hexosaminidase that has the enzymatic activity of the parent, i.e. the capacity of catalyzing the hydrolysis of β-1,6-glycosidic linkages of N-acetyl-glucosamine polymers (hexosaminidase activity). In one embodiment, the hexosaminidase activity of the variant is increased with reference to the parent hexosaminidase, e.g. the polypeptide of SEQ ID NO: 10.

Variant nomenclature: In describing enzyme variants herein, the nomenclature described below is adapted for ease of reference. The accepted IUPAC single letter amino acid abbreviation is generally employed.

Substitutions: For an amino acid substitution, the following nomenclature is used: Original amino acid, position, substituted amino acid. Accordingly, the substitution of threonine at position 226 with alanine is designated as “T226A”. Multiple mutations may be separated by addition marks (“+”), e.g., “G205R+S411F”, or by a comma, e.g. “G205R, S411F”, representing substitutions at positions 205 and 411 of glycine (G) with arginine (R) and serine(S) with phenylalanine (F), respectively.

Deletions: For an amino acid deletion, the following nomenclature is used: Original amino acid, position, *. Accordingly, the deletion of glycine at position 195 is designated as “G195*”.

Insertions: For an amino acid insertion, the following nomenclature is used: Original amino acid, position, original amino acid, inserted amino acid. Accordingly, the insertion of lysine after glycine at position 195 is designated “G195GK”. An indication of an insertion at a particular position is understood as being an insertion after the original amino acid residue. For example, an “insertion at position 195” is understood to be an insertion after the original residue in position 195.

Multiple alterations: Variants comprising multiple alterations are separated by addition marks (“+”) or commas, e.g., “R170Y+G195E” or “R170Y,G195E” representing a substitution of arginine and glycine at positions 170 and 195 with tyrosine and glutamic acid, respectively.

Different alterations: Where different alterations can be introduced at a position, the different alterations may be separated by a comma, e.g., “R170Y,E” represents a substitution of arginine at position 170 with tyrosine or glutamic acid. Thus, “Y167G,A+R170G,A” designates the following variants: “Y167G+R170G”, “Y167G+R170A”, “Y167A+R170G”, and “Y167A+R170A”.

Medical device: The term “medical device” is intended to refer broadly to any kind of medical or dental device, instrument or equipment which comes into contact with a patient, where the patient may be a human or an animal. Medical devices include devices, instruments, tools, apparatus and equipment used in medicine or surgery, e.g. for a diagnosis or an operation, including in dentistry and veterinary medicine, and including those than can be cold sterilized, soaked or washed and then heat sterilized, or which otherwise may benefit from cleaning as described herein. Non-limiting examples of medical devices include surgical and diagnostic instruments such as trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes, arthoscopes and related equipment, and the like, or combinations thereof. Other examples of medical devices include surgical instruments such as scalpels, hemostats, Kocher forceps and tracheotomes.

The medical device may be an indwelling device, for example a catheter such as a central venous catheter, intravascular catheter, urinary catheter, Hickman catheter, peritoneal dialysis catheter or endrotracheal catheter; or a device such as a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector or a needleless connector.

The term “at least the part of the medical device” is to be understood as a part of device being in contact with the patient, or the interior of e.g. an endoscope which is in contact with fluids or samples which have been in contact with the patient.

Wash liquor: The term “wash liquor” is intended to mean the solution or mixture of water and/or another solvent and at least one cleaning component e.g. a surfactant, and which is used for cleaning a surface of at least a part of a medical device.

### DETAILED DESCRIPTION OF THE INVENTION

The inventors have surprisingly found that washing of a medical device with two or more enzymes selected from the group consisting of a protease, an enzyme having DNase activity and an enzyme having hexosaminidase activity gives a synergistic wash performance particularly with regard to reducing/removing biofilm from e.g. medical devices.

Thorough cleaning of medical devices, e.g., endoscopes or surgical instruments, is crucial to ensure that medical devices are properly disinfected and sterilized. Residual inorganic and organic materials e.g. biofilms will interfere with the effectiveness of disinfection and sterilization processes. As a result, infectious pathogens may still live and exist on the medical devices after wash, disinfection and sterilization, and may be transmitted to other patients.

The present invention addresses these challenges by providing a cleaning composition comprising two or more different enzymes that has been found to have synergistic wash performance for cleaning a medical device, and which can thereby provide deep cleaning benefit on medical devices and greatly reduce the risk of transmitting infectious pathogens from one patient to another.

Thus, one aspect of the invention relates to a method of cleaning an item e.g. a medical device, comprising the steps of:
(a) contacting the medical device with a wash liquor comprising two or more enzymes selected from the group consisting of a protease, an enzyme having DNase activity and an enzyme having hexosaminidase activity; and optionally
(b) rinsing the medical device.

The wash performance of the method or the composition of the invention can be evaluated by measuring the percentage of removed biofilms present on the medical device as compared to that washed without said enzymes, e.g. as set forth in Example 1. In one embodiment, the amount of biofilm existing on the medical device after cleaning is reduced by at least 1%, e.g. at least 2%, at least 5%, at least 10%, at least 15%, at least 20%, at least 30%, or at least 40% compared to cleaning with a wash liquor without said two or more enzymes.

For the purpose of e.g. facilitating the cleaning performance or disinfection effect, the present method may further comprise a step of pre-treating the medical device with a disinfectant before the above step a). Suitable disinfectants may include, for example, peracetic acids, hydrogen peroxides, potassium permanganate, chlorine dioxide and ethanol. The pre-treatment may be carried out by soaking or contacting the medical device with a solution containing said disinfectant.

In one embodiment, the medical device is rinsed with water. In another embodiment, the medical device is rinsed with a solution comprising a disinfectant as described above.

In one embodiment, the medical device is an endoscope. Examples of such endoscopes may be cystoscopes, nephroscopes, bronchoscopes, laryngoscopes, otoscopes, arthroscopses, laparoscopes, gastrointestinal endoscopes or the like.

In one embodiment, the medical device is a surgical instrument, such as a scalpel, a hemostat, a forceps, scissors, a retractor, a tracheotome and a clamp.

In another embodiment, the medical device is a dental instrument that is used by e.g. dentists to remove teeth, or to identify and select for dental treatments. Examples of such dental instruments may be scalers, curettes, serrated cotton pliers, dental mirrors or the like.

Biofilm present on medical devices can be found among many microorganisms, e.g. the following species: Enterobacteriacae (e.g., Escherichia coli), Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538. In one embodiment, the medical device may be soiled with biofilm produced by or partly produced by microorganisms selected from following group consisting of Enterobacteriacae (e.g., Escherichia coli), Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538, Streptococcus sp. (e.g., S. pyogenes, S. agalacticae or S. pneumoniae), Haemophilus influenzae, Pseudomonas aeruginosa, Clostridium perfringens, Chlamydia trachomatis, Candida albicans, Bacillus anthracis, or combinations thereof.

The concentration of each enzyme in the wash liquor may be in the range of 0.0005-100 ppm enzyme protein, such as in the range of 0.001-50 ppm, in the range of 0.005-20 ppm, in the range of 0.01-10 ppm, in the range of 0.1-15 ppm, or in the range of 0.05-5 ppm enzyme protein.

Another aspect of the present invention provides a composition for cleaning a medical device, wherein said cleaning composition comprises a surfactant and two or more enzymes selected from the group consisting of a protease, an enzyme having DNase activity and an enzyme having hexosaminidase activity.

The amount of each enzyme of the invention to be included in the cleaning composition is at least 0.0001 mg of enzyme protein per gram of the composition, such as at least 0.001 mg of enzyme protein, at least 0.006 mg of enzyme protein, at least 0.008 mg of enzyme protein, at least 0.01 mg of enzyme protein, at least 0.1 mg of enzyme protein, at least 0.5 mg of enzyme protein, at least 1 mg of enzyme protein, at least 2 mg of enzyme protein, at least 5 mg of enzyme protein, at least 10 mg of enzyme protein, or at least 20 mg of enzyme protein.

Another aspect of the present invention relates to use of an enzyme composition for cleaning a medical device, e.g. an endoscope, a surgical instrument or a dental instrument, wherein the enzyme composition comprises two or more enzymes selected from the group consisting of a protease, an enzyme having DNase activity and an enzyme having hexosaminidase activity.

Undesirable microorganisms (e.g. pathogenic bacteria) are not only present in medical equipment but may also appear in biological manufacturing equipment (e.g., food and beverage manufacturing, and biotech manufacturing such as enzyme fermentation processes), e.g., fermentation and storage tanks, bioreactors, ultrafilters or ultrafiltration membranes, pipelines and other equipment. Such equipment is typically cleaned by cleaning-in-place (CIP) methods, where cleaning is performed without removing or disassembling piping or other equipment.

Therefore, in another aspect the present invention provides a CIP (cleaning-in-place) cleaner comprising two or more enzymes selected from the group consisting of a protease, an enzyme having DNase activity and an enzyme having hexosaminidase activity.

The present invention further relates to a CIP cleaning method, comprising performing cleaning-in-place using two or more enzymes selected from the group consisting of a protease, an enzyme having DNase activity and an enzyme having hexosaminidase activity; and optionally including a rinse step.

### Enzymes of the Invention

In one embodiment, the DNase according to the present invention is selected from any of the enzyme classes E.C.3.1, preferably E.C.3.1.21, e.g. such as E.C.3.1.21.X, where X=1, 2, 3, 4, 5, 6, 7, 8 or 9, or e.g. Deoxyribonuclease I, Deoxyribonuclease IV, Type I site-specific deoxyribonuclease, Type II site-specific deoxyribonuclease, Type III site-specific deoxyribonuclease, CC-preferring endo-deoxyribonuclease, Deoxyribonuclease V, T (4) deoxyribonuclease II, T(4) deoxyribonuclease IV or E.C. 3.1.22.Y where Y=1, 2, 4 or 5, e.g. Deoxyribonuclease II, Aspergillus deoxyribonuclease K(1), Crossover junction endo-deoxyribonuclease, Deoxyribonuclease X.

Preferably, the polypeptide having DNase activity is obtained from a microorganism and the DNase is a microbial enzyme. The DNase is preferably of fungal or bacterial origin, or a variant of a DNase of fungal or bacterial origin. In one embodiment a DNase obtained from a fungus e.g. Aspergillus oryzae, or a variant thereof, is used.

Suitable bacterial DNases may, for example, be obtained from species of Bacillus and related genera (cf. Patel and Gupta, Int. J. Syst. Evol. Microbiol. 2020; 70:406-438, who proposed six new Bacillaceae genera from species formerly classified as belonging to the genus Bacillus), e.g. from Bacillus, Cytobacillus, Metabacillus, Alkalihalobacillus, Rossellomorea or Mesobacillus. Examples of species from which DNases may be obtained include Bacillus licheniformis, Bacillus subtilis, Sutcliffiella horikoshii, Cytobacillus horneckiae, Metabacillus indicus, Alkalihalobacillus algicola, Rossellomorea vietnamensis, Alkalihalobacillus hwajinpoensis, Mesobacillus campisalis, Bacillus idriensis, Rossellomorea marisflavi and Bacillus luciferensis. Preferred bacterial DNases include those obtained from Metabacillus indicus (previously known as Bacillus cibi) and variants thereof.

In one embodiment, the DNase of the present invention may be a polypeptide having DNase activity and a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 1. The enzyme may e.g. differ by up to 20 amino acids, up to 15 amino acids or up to 10 amino acids, e.g., 1, 2, 3, 4, 5, 6, 7, 8, 9, or 10, from the mature polypeptide of SEQ ID NO: 1.

In one embodiment, the DNase of the present invention may be a polypeptide having DNase activity and a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 2. The enzyme may e.g. differ by up to 20 amino acids, up to 15 amino acids or up to 10 amino acids, e.g., 1, 2, 3, 4, 5, 6, 7, 8, 9, or 10, from the mature polypeptide of SEQ ID NO: 2.

In one embodiment, the DNase of the present invention may be a polypeptide having DNase activity and a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 3. The enzyme may e.g. differ by up to 20 amino acids, up to 15 amino acids or up to 10 amino acids, e.g., 1, 2, 3, 4, 5, 6, 7, 8, 9, or 10, from the mature polypeptide of SEQ ID NO: 3.

In one embodiment, the DNase of the present invention may be a polypeptide having DNase activity and a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the mature polypeptide of SEQ ID NO: 4. The enzyme may e.g. differ by up to 20 amino acids, up to 15 amino acids or up to 10 amino acids, e.g., 1, 2, 3, 4, 5, 6, 7, 8, 9, or 10, from the mature polypeptide of SEQ ID NO: 4.

In one embodiment, the DNases of the invention preferably belong to the NUC1 group of DNases. The NUC1 group of DNases comprises polypeptides which in addition to having DNase activity, may comprise one or more of the motifs [T/D/S][G/N]PQL (SEQ ID NO: 23), corresponding to positions 116 to 120 of SEQ ID NO: 4; [F/L/Y/I]A[N/R]D[L/I/P/V] (SEQ ID NO: 24), corresponding to positions 111 to 115 of SEQ ID NO: 4; and C[D/N]T[A/R] (SEQ ID NO: 25), corresponding to positions 44 to 47 of SEQ ID NO: 4. The DNases of this group preferably further comprise a NUC1_A domain [D/Q][I/V]DH (SEQ ID NO: 26), corresponding to positions 85 to 88 of SEQ ID NO: 4.

In one embodiment, the DNases of the invention preferably belong to the group of DNases comprised in the GYS-clade, which are group of DNases on the same branch of a phylogenetic tree having both structural and functional similarities. These NUC1 and/or NUC1_A DNases comprise the conservative motifs [D/M/L][S/T]GYSR[D/N] (SEQ ID NO: 27) and/or ASXNRSKG (SEQ ID NO: 28) and share similar structural and functional properties. In the present invention, motif [D/M/L][S/T]GYSR[D/N] (SEQ ID NO: 27) corresponds to positions 26 to 32 of SEQ ID NO: 4, and motif ASXNRSKG (SEQ ID NO: 28) corresponds to positions 125 to 132 of SEQ ID NO: 4. The DNases of the GYS-clade are preferably obtained from species of Bacillus and related genera (cf. Patel and Gupta, supra).

In one embodiment, the polypeptide having DNase activity according to the invention comprises one or more (e.g., two, three, four, five or six) of the motifs selected from the group consisting of [T/D/S][G/N]PQL (SEQ ID NO: 23), [F/L/Y/I]A[N/R]D[L/I/P/V] (SEQ ID NO: 24), C[D/N]T[A/R] (SEQ ID NO: 25), [D/Q][I/V]DH (SEQ ID NO: 26), [D/M/L][S/T]GYSR[D/N] (SEQ ID NO: 27) and ASXNRSKG (SEQ ID NO: 28), wherein the polypeptide has at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4.

In one embodiment, the polypeptide having DNase activity according to the invention comprises one or both motifs [D/M/L][S/T]GYSR[D/N] (SEQ ID NO: 27) and ASXNRSKG (SEQ ID NO: 28), and optionally further comprises one or more (e.g., two, three, four) of the motifs selected from the group consisting of [T/D/S][G/N]PQL (SEQ ID NO: 23), [F/L/Y/I]A[N/R]D[L/I/P/V] (SEQ ID NO: 24), C[D/N]T[A/R] (SEQ ID NO: 25), [D/Q][I/V]DH (SEQ ID NO: 26), wherein the polypeptide has at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4.

In one embodiment, the enzyme having DNase activity is a variant of SEQ ID NO: 2, comprising one or more alterations selected from the group consisting of S26*, D32E,Q, V35I, K36C, H, G37R, F43W, D46G, A55I, N68D, S69V, A76I, K82S, T, P84D, T, K86E,G,L,N,Q,T,V,Y, A91R, L92E, K95I, P97E,N, A101E, Q102E, K105N,G,Q,T,D, A111P, F112Y,W, S115T, V127T, L129K, N133Q, G137R, V138C, N140H, G141Q,R, S144E, N146A, K147N,E, V148I, A149D,E,F, Q150D,E, P153D,V, S154E, K155E,F,L,S,T, Q157D,E, Q158D, T159Q, K160D, G161R, T170Q, A172D,E,H,R, G18I N, K185*, V187N, Y, N191*, K192A,I, D197K,S, G199Q, Q208V, E211Y,T,P, N213S, N214D, N217A and Y218D,E, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

In one embodiment, the enzyme having DNase activity is a variant of SEQ ID NO: 2, comprising the substitution D32E and one or more alterations selected from the group consisting of V35I, S69V, K86E, Q102E, K105N, A111P, S115T, V127T, G137R, K147N, Q150E, K155E, T159Q, G161R, A172D, G181N, V187N, K192I, K192A, Q208V and N217A, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

In one embodiment, the enzyme having DNase activity is a variant of SEQ ID NO: 2, comprising the substitution V35I and one or more alterations selected from the group consisting of D32E, S69V, K86E, Q102E, K105N, A111P, S115T, V127T, G137R, K147N, Q150E, K155E, T159Q, G161R, A172D, G181N, V187N, K192I, K192A, Q208V and N217A, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

In one embodiment, the enzyme having DNase activity is a variant of SEQ ID NO: 2, comprising the substitution K105N and one or more alterations selected from the group consisting of D32E, V35I, S69V, K86E, Q102E, A111P, S115T, V127T, G137R, K147N, Q150E, K155E, T159Q, G161R, A172D, G181N, V187N, K192I, K192A, Q208V and N217A, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

In one embodiment, the enzyme having DNase activity is a variant of SEQ ID NO: 2, comprising the substitution A111P and one or more alterations selected from the group consisting of D32E, V35I, S69V, K86E, Q102E, K105N, S115T, V127T, G137R, K147N, Q150E, K155E, T159Q, G161R, A172D, G181N, V187N, K192I, K192A, Q208V and N217A, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

In one embodiment, the enzyme having DNase activity is a variant of SEQ ID NO: 2, comprising at least two substitutions selected from D32E, V35I, S69V,Q102E, K105N, A111P, S115T, G161R and G181N, and further comprising one or more alterations selected from the group consisting of K65E, K67A, K86E, V127T, G137R, K147N, Q150E, K155E, T159Q, A172D, V187N, K192I, K192A, Q208V and N217A, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

In one embodiment, the DNase variant comprises or consists of SEQ ID NO: 2 with the substitutions S69V+Q102E+K105N+A111P+S115T+Q150E+G161R+G181N+V187N+K192I. In another embodiment, the DNase variant comprises or consists of SEQ ID NO: 2 with the substitutions D32E+V35I+S69V K86E+Q102E+K105N+A111P+S115T+V127T+G137R+K147N+Q150E+K155E T159Q+G161R+A172D+G181N+V187N+K192A+Q208V+N217A.

In another embodiment, the enzyme having DNase activity is a variant of SEQ ID NO: 4, comprising one or more alterations at positions 1, 13, 22, 25, 27, 33, 39, 42, 56, 57, 59, 65, 76, 77, 109, 116, 127, 144, 147, 149, 167, 175, and 181 of SEQ ID NO: 4, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 4. In a preferred embodiment, the DNase variant comprises one or more of the substitutions T1I, S13Y, T22P, S25P, S27L, L33K, S39P, S42G, D56I, S57W, S59V, T65V, V76L, T77Y, Q109R, S116D, T127V, S144P, A147H, G149N, S167L, G175D and S181L of SEQ ID NO: 4.

In a particular embodiment, the DNase variant comprises or consists of SEQ ID NO: 4 with the substitutions T1I, S13Y, T22P, S27L, L33K, S39P, S42G, D56I, S57W, S59V, T65V, V76L, Q109R, S116D, T127V, S144P, A147H, S167L and G175D. In another embodiment, the DNase variant comprises or consists of SEQ ID NO: 4 with the following substitutions: T1I+S13Y+T22P+S25P+S27L+S39P+S42G+S57W+S59V+T65V+V76L+T77Y+Q109R+S116D+S144P+A147H+G149N+S167L+G175D+S181L.

In one aspect, the enzyme having hexosaminidase activity according to the invention, which is useful for thorough cleaning of medical devices, is selected from the polypeptides having at least 60%, such as at least 65%, such as at least 70%, such as at least 75%, such as at least 80%, such as at least 85%, or such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the mature polypeptide of SEQ ID NO: 10, 11, 19, 20, 21 or 22, and preferably wherein the hexosaminidase has N-acetylglucosaminidase activity and/or β-N-acetylglucosamininidase activity.

In one aspect, the enzyme having hexosaminidase activity according to the invention is obtained from the Terribacillus clade (e.g. from Terribacillus saccharophilus) and has at least 60%, such as at least 65%, such as at least 70%, such as at least 75%, such as at least 80%, such as at least 85%, or such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the mature polypeptide of SEQ ID NO: 10 or SEQ ID NO: 19, and preferably wherein the hexosaminidase has N-acetylglucosaminidase activity and/or B-N-acetylglucosamininidase activity.

In one aspect, the enzyme having hexosaminidase activity according to the invention is obtained from Lactobacillus, e.g. Lactobacillus paraplantarum and has at least 60%, such as at least 65%, such as at least 70%, such as at least 75%, such as at least 80%, such as at least 85%, or such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the mature polypeptide of SEQ ID NO: 11, and preferably wherein the hexosaminidase has N-acetylglucosaminidase activity and/or β-N-acetylglucosamininidase activity.

In one aspect, the enzyme having hexosaminidase activity according to the invention is obtained from Staphylococcus, e.g. Staphylococcus cohnii, and has at least 60%, such as at least 65%, such as at least 70%, such as at least 75%, such as at least 80%, such as at least 85%, or such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the mature polypeptide of SEQ ID NO: 20, and preferably wherein the hexosaminidase has N-acetylglucosaminidase activity and/or B—N-acetylglucosamininidase activity.

In one aspect, the enzyme having hexosaminidase activity according to the invention is obtained from Staphylococcus, e.g. Staphylococcus fleurettii, and has at least 60%, such as at least 65%, such as at least 70%, such as at least 75%, such as at least 80%, such as at least 85%, or such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the mature polypeptide of SEQ ID NO: 21, and preferably wherein the hexosaminidase has N-acetylglucosaminidase activity and/or B—N-acetylglucosamininidase activity.

In one aspect, the enzyme having hexosaminidase activity according to the invention is obtained from Aggregatibacter, e.g. Aggregatibacter actinomycetemcomitans, and has at least 60%, such as at least 65%, such as at least 70%, such as at least 75%, such as at least 80%, such as at least 85%, or such as at least 90%, such as at least 91%, such as at least 92%, such as at least 93%, such as at least 94%, such as at least 95%, such as at least 96%, such as at least 97%, such as at least 98%, such as at least 99% or 100% sequence identity to the mature polypeptide of SEQ ID NO: 22, and preferably wherein the hexosaminidase has N-acetylglucosaminidase activity and/or B—N-acetylglucosamininidase activity.

In one aspect, the enzyme having hexosaminidase activity according to the invention may comprise the structural domains of Glyco_hydro_20 e.g. GH20. Polypeptides comprising a GH20 domain may comprise several motifs. One example is motif GXDE (SEQ ID NO: 12) situated in positions corresponding to positions 158 to 161 in SEQ ID NO: 10). Residues D and E are the key catalytic residues of GH20 (positions 160 and 161 in SEQ ID NO: 10). The GH20 polypeptides can be separated into multiple distinct sub-clusters, or clades as set forth in WO 2017/186943 (incorporated herein by reference). Examples of specific domains are listed below.

A further domain is termed IAS and polypeptides of this domain are in addition to having hexosaminidase activity e.g. PNAG activity, characterized by comprising certain motifs e.g. [EQ][NRSHA][YVFL][AGSTC][IVLF][EAQYN][SN] (SEQ ID NO: 13), corresponding to ESYAIAS at position 44 to 50 of SEQ ID NO: 10.

Another domain, preferably shared by the hexosaminidase polypeptides of the invention, is termed WND. Polypeptides of this domain preferably comprise a GH20 domain, are of bacterial origin and are in addition to having PNAG activity, characterized by comprising certain motifs. The polypeptides having the WND domain may comprise the motif [VIM][LIV]G[GAV]DE[VI][PSA] (SEQ ID NO: 14), corresponding to positions 156 to 163 of SEQ ID NO: 10, where G (corresponding to position 158 of SEQ ID NO: 10) is fully conserved in Terribacillus clade and residues D and E are the key catalytic residues of GH20 (positions 160 and 161 in SEQ ID NO: 10). Another motif which may be comprised by the polypeptides of the invention is WND [SQR][IVL][TLVM] (SEQ ID NO: 15), positions 193 to 198 in SEQ ID NO: 10, where W (position 193 in SEQ ID NO: 10) is part of the active site pocket and putatively involved in binding of the N-acetyl group of the PNAG substrate.

The polypeptides of the Terribacillus clade may be further subdivided in a clade termed QSTL, which comprises WND domain polypeptides of bacterial origin having PNAG activity. The polypeptides of the clade comprise the motif example QSTL (SEQ ID NO: 16), corresponding to positions 216 to 219 of SEQ ID NO: 10, where all four amino acids are fully conserved in QSTL clade and putatively involved in substrate binding. Another motif which may be comprised by the polypeptides of the QSTL clade is NKFFY (SEQ ID NO: 17), 273 to 277 in SEQ ID NO: 10. A further motif which may be comprised by the polypeptides of the QSTL clade is NLD[DR]S (SEQ ID NO: 18), 204 to 208 in SEQ ID NO: 10.

In one aspect, the polypeptides having hexosaminidase activity according to the invention comprise the GXDE (SEQ ID NO: 12) motif. In one aspect, the polypeptides having hexosaminidase activity according to invention comprise the [EQ][NRSHA][YVFL][AGSTC][IVLF][FAQYN][SN] (SEQ ID NO: 13) motif. In one aspect, the polypeptides having hexosaminidase activity according to the invention comprise the motif [VIM][LIV]G[GAV]DE[VI][PSA] (SEQ ID NO: 14). In one aspect, the polypeptides having hexosaminidase activity according to the invention comprise the motif WND [SQR][IVL][TLVM] (SEQ ID NO: 15). In one aspect, the polypeptides having hexosaminidase activity according to the invention comprise the motif QSTL (SEQ ID NO: 16). In one aspect, the polypeptides having hexosaminidase activity according to the invention comprise the motif NKFFY (SEQ ID NO: 17). In one aspect, the polypeptides having hexosaminidase activity according to the invention comprise the motif NLD[DR]S (SEQ ID NO: 18).

In one aspect, the polypeptide having hexosaminidase activity according to the invention comprises one or more of the motif(s) GXDE (SEQ ID NO: 12), [EQ][NRSHA][YVFL][AGSTC][IVLF][EAQYN][SN] (SEQ ID NO: 13), [VIM][LIV]G[GAV]DE[VI][PSA] (SEQ ID NO: 14), WND [SQR][IVL][TLVM] (SEQ ID NO: 15), QSTL (SEQ ID NO: 16), NKFFY (SEQ ID NO: 17), NLD[DR]S (SEQ ID NO: 18), wherein the polypeptide has hexosaminidase activity, preferably N-acetylglucosaminidase activity and/or β-N-acetylglucosamininidase activity, and wherein the polypeptide has at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10, 11, 19, 20, 21 or 22.

In one aspect, the polypeptide having hexosaminidase activity according to the invention comprises two, three, four, five, six or all seven of the motifs GXDE (SEQ ID NO: 12), [EQ][NRSHA][YVFL][AGSTC][IVLF][EAQYN][SN] (SEQ ID NO: 13), [VIM][LIV]G[GAV]DE[VI][PSA] (SEQ ID NO: 14), WND [SQR][IVL][TLVM] (SEQ ID NO: 15), QSTL (SEQ ID NO: 16), NKFFY (SEQ ID NO: 17), NLD[DR]S (SEQ ID NO: 18), wherein the polypeptide has hexosaminidase activity, preferably N-acetylglucosaminidase activity and/or β-N-acetylglucosamininidase activity, and wherein the polypeptide has at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10, 11, 19, 20, 21 or 22.

In another aspect, the hexosaminidase is a variant of polypeptide of SEQ ID NO:10, comprising one or more alterations at positions 3, 15, 49, 59, 163, 186, 225, 227, 232, 235, 252, 260, 272, 279, 281, 308, 309 and 312 of SEQ ID NO: 10, wherein the variant has hexosaminidase activity, preferably N-acetylglucosaminidase activity and/or β-N-acetylglucosamininidase activity, and wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 10. More preferably, the variant has one or more of the motif(s) GXDE (SEQ ID NO: 12), [EQ][NRSHA][YVFL][AGSTC][IVLF][EAQYN][SN] (SEQ ID NO: 13), [VIM][LIV]G[GAV]DE[VI][PSA] (SEQ ID NO: 14), WND [SQR][IVL][TLVM] (SEQ ID NO: 15), QSTL (SEQ ID NO: 16), NKFFY (SEQ ID NO: 17), NLD[DR]S (SEQ ID NO: 18).

In one embodiment, the hexosaminidase according to the present invention is a variant of polypeptide of SEQ ID NO:10, comprising one or more of the substitutions Q3I, H15Y, A49W, N59E, S163P, S186R, S225G, N227T, E232D, G235W, N252P, N260Q, H272V, S279D, Y281P, K308Q, K309E and K312Q of SEQ ID NO:10, wherein the variant has hexosaminidase activity, preferably N-acetylglucosaminidase activity and/or β-N-acetylglucosamininidase activity, and wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 10. More preferably, the variant has one or more of the motif(s) GXDE (SEQ ID NO: 12), [EQ][NRSHA][YVFL][AGSTC][IVLF][EAQYN][SN] (SEQ ID NO: 13), [VIM][LIV]G[GAV]DE[VI][PSA] (SEQ ID NO: 14), WND [SQR][IVL][TLVM] (SEQ ID NO: 15), QSTL (SEQ ID NO: 16), NKFFY (SEQ ID NO: 17), NLD[DR]S (SEQ ID NO: 18).

In one specific embodiment, the hexosaminidase variant comprises or consists of SEQ ID NO: 10 with the substitutions Q3I, H15Y, A49W, N59E, S163P, S186R, S225G, N227T, E232D, G235W, N252P, N260Q, H272V, S279D, Y281P, K308Q, K309E and K312Q.

Proteases suitable for the present invention may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus and related genera (cf. Patel and Gupta, supra). Examples of Bacillus species from which subtilases have been derived include Bacillus lentus, Bacillus alcalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V199I, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

In some embodiments, a preferred protease according to the present invention may be a protease having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 5, 6, 7, 8, 9 or 29.

In another embodiment, a preferred protease according to the present invention may be a variant of the polypeptide of SEQ ID NO: 6 comprising an alteration at one or more positions corresponding to positions 3, 4, 9, 15, 43, 68, 76, 99, 101, 103, 104, 160, 167, 170, 194, 199, 205, 206, 209, 217, 218, 222, 245, 261 and 262, wherein position numbers correspond to the positions of SEQ ID NO: 5, wherein each alteration is independently a substitution, deletion or insertion, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 6.

In one embodiment, the protease of the invention is a variant of the polypeptide of SEQ ID NO: 6, comprising one or more substitutions selected from the group consisting of: S3T, V4I, S9E, S9R, A15T, V68A, N76D, S99D, S99G, S99A, S99SE, S101E, S101N, S101R, S103A, V104I, G160S, Y167A, R170S, A194P, V199M, V205I, Q206L, Y209W, L217D, L217Q, N218D, M222S, Q245R, N261W and L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, at least 99%, but less than 100% sequence identity to SEQ ID NO: 6.

In one embodiment, the protease of the invention is a variant of the polypeptide of SEQ ID NO: 6, comprising one or more substitutions selected from the group consisting of: S3T, V4I, S9E, S9R, A15T, T22A, N43R, V68A, N76D, S87N, S99D, S99G, S99A, S99SE, S101E, S101N, S101R, S103A, V104I, G118M, S128Q, G160S, Y167A, R170S, N184E, A194P, V199M, V205I, Q206L, Y209W, L217D, L217Q, N218D, M222S, Q245R, S259D, N261W and L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, at least 99%, but less than 100% sequence identity to SEQ ID NO: 6.

In one embodiment, the protease of the invention is a variant of the polypeptide of SEQ ID NO: 6, comprising the substitution S87N, wherein the variant has protease activity and wherein the position corresponds to the position of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6.

In one embodiment, the protease of the invention is a variant of the polypeptide of SEQ ID NO: 6, comprising the substitutions Y167A+R170S+A194P, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6.

In one embodiment, the protease of the invention is a variant of the polypeptide of SEQ ID NO: 6, comprising the substitutions S9E+N43R+N76D+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6.

In one embodiment, the protease of the invention is a variant of the polypeptide of SEQ ID NO: 6, comprising the substitutions S3T+N43R+N76D+S87N+G118M+S128Q+N184E+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6.

In one embodiment, the protease of the invention is a variant of the polypeptide of SEQ ID NO: 6, comprising the substitutions T22A+N43R+S87N+V205L+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6.

In one embodiment, the protease of the invention is a variant of the polypeptide of SEQ ID NO: 8, comprising the substitutions A68S+T77N+T78I+G127S+A128P+G165Q+N184Q+A202V+N217S+S258P, wherein position numbers correspond to the positions of SEQ ID NO: 8, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO:8.

In one embodiment, the protease of the invention is a variant comprising a substitution at one or more positions corresponding to positions 171, 173, 175, 179 or 180 of SEQ ID NO: 1 of WO2004/067737, wherein the variant has protease activity and has a sequence identity of at least 80%, at least 85%, at least 90%, at least 91%, at least 92%, at least 93%, at least 94%, at least 95%, at least 96%, at least 97%, at least 98%, or at least 99%, but less than 100% to SEQ ID NO: 1 of WO2004/067737.

In one embodiment, the protease of the invention is a variant comprising one or more substitutions compared to a parent protease, selected from the group consisting of X3V, X9[E,R], X22[R,A], X43R, X61[E,D], X62[E,D], X76[D], X87N, X101[E,G,D,N,M], X103A, X104I, X118[V,R,M], X120V, X128[A,L,S,Q], X129Q, X130A, X160D, X184[E,D], X185[E,D], 188[E,D], X191N, X194P, X205I, X206L, X209W, X216V, X217[Q,D,E], X218[D,E,S], X232V, X245R, X248D, X256[E,D], X259[E,D], X261[E,D,W] and X262[E,D], wherein position numbers correspond to the positions of BPN′ (SEQ ID NO: 5), wherein “X” represents any amino acid residue present in the specified position in the parent protease, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or SEQ ID NO: 6.

In one embodiment, the protease of the invention is a variant comprising any of the following substitution sets compared to a parent protease, wherein the parent protease has the amino acid sequence of SEQ ID NO: 5 or SEQ ID NO: 6 or has at least 80% sequence identity to SEQ ID NO: 5 or SEQ ID NO: 6, wherein position numbers correspond to the positions of BPN′ (SEQ ID NO: 5), wherein “X” represents any amino acid residue present in the specified position in the parent protease, and wherein the substitution set is selected from the group consisting of:
i. X9R+X15T+X68A+X218D+X245R,
ii. X9R+X15T+X68A+X245R,
iii. X61E+X194P+X205I+X261D,
iv. X61D+X205I+X245R,
V. X61E+X194P+X205I+X261D,
vi. X87N+X118V+X128L+X129Q+X130A,
vii. X87N+X101M+X118V+X128L+X129Q+X130A,
viii. X76D+X87R+X118R+X128L+X129Q+X130A,
ix. X22A+X62D+X101G+X188D+X232V+X245R,
x. X103A+X104I,
xi. X22R+X101G+X232V+X245R,
xii. X103A+X104I+X156D,
xiii. X103A+X104I+X261E,
xiv. X62D+X245R,
XV. X101N+X128A+X217Q,
xvi. X101E+X217Q,
xvii. X101E+X217D,
xviii. X9E+X43R+X262E,
xix. X76D+X43R+X209W,
xx. X205I+X206L+X209W,
xxi. X185E+X188E+X205I,
xxii. X256D+X261W+X262E,
xxiii. X191N+X209W,
xxiv. X261E+X262E,
xxv. X261E+X262D, and
xxvi. X167A+X170S+X194P,
wherein the protease variant has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or 6.

In one embodiment, the protease variant of the invention comprises the amino acid sequence of SEQ ID NO: 6 with the substitutions Y167A+R170S+A194P, wherein the variant has protease activity and wherein the positions correspond to the positions of SEQ ID NO: 5.

In one embodiment, the protease variant of the invention comprises the amino acid sequence of SEQ ID NO: 6 with the substitutions S9E+N43R+N76D+V205I+Q206L+Y209W+S259D+N261W+L262E, wherein the variant has protease activity and wherein position numbers correspond to the positions of SEQ ID NO: 5.

In one embodiment, the protease of the invention is an enzyme having or consisting of the amino acid sequence of SEQ ID NO: 6, 7, 8, 9 or 29.

In one embodiment, the wash liquor or the cleaning composition comprises a protease and an enzyme having DNase activity. In one embodiment, the wash liquor or the cleaning composition comprises a protease and an enzyme having hexosaminidase activity. In another embodiment, the wash liquor or the cleaning composition comprises an enzyme having DNase activity and an enzyme having hexosaminidase activity. In yet another embodiment, the wash liquor or the cleaning composition comprises a protease, an enzyme having DNase activity and an enzyme having hexosaminidase activity.

Various parent proteases are suitable for making the variants suitable together with a DNase and/or a hexosaminidase for obtaining the beneficial effects described in the present invention e.g. significantly improved reduction of biofilm. It is clear to the skilled artisan that the protease may comprise additional substitutions.

Thus, in some embodiments, the wash liquor or the cleaning composition comprises: a protease having SEQ ID NO: 6 with the substitutions Y167A+R170S+A194P, wherein position numbers are based on the numbering of SEQ ID NO: 5; a DNase having SEQ ID NO: 4 with the substitutions T1I, S13Y, T22P, S27L, L33K, S39P, S42G, D56I, S57W, S59V, T65V, V76L, Q109R, S116D, T127V, S144P, A147H, S167L and G175D; and a hexosaminidase having SEQ ID NO: 10, 11, 19, 20, 21 or 22.

In some embodiments, the wash liquor or the cleaning composition comprises: a protease having SEQ ID NO: 6 with the substitutions Y167A+R170S+A194P, wherein position numbers correspond to the positions of SEQ ID NO: 5; a DNase having SEQ ID NO: 3 or 4; and a hexosaminidase having SEQ ID NO: 10, 11, 19, 20, 21 or 22.

In some embodiments, the wash liquor or the cleaning composition comprises: a protease having SEQ ID NO: 8; a DNase having SEQ ID NO: 4 with the substitutions T1I, S13Y, T22P, S27L, L33K, S39P, S42G, D56I, S57W, S59V, T65V, V76L, Q109R, S116D, T127V, S144P, A147H, S167L and G175D; and a hexosaminidase having SEQ ID NO: 10 with the substitutions Q3I, H15Y, A49W, N59E, S163P, S186R, S225G, N227T, E232D, G235W, N252P, N260Q, H272V, S279D, Y281P, K308Q, K309E and K312Q.

In some embodiments, the wash liquor or the cleaning composition comprises: a protease having SEQ ID NO: 8 with the substitutions A68S+T77N+T78I+G127S+A128P+G165Q+N184Q+A202V+N217S+S258P, wherein position numbers correspond to the positions of SEQ ID NO: 8; and a hexosaminidase having SEQ ID NO: 10 with the substitutions Q3I, H15Y, A49W, N59E, S163P, S186R, S225G, N227T, E232D, G235W, N252P, N260Q, H272V, S279D, Y281P, K308Q, K309E and K312Q.

In some embodiments, the wash liquor or the cleaning composition comprises a DNase having SEQ ID NO: 4 with the substitutions T1I, S13Y, T22P, S27L, L33K, S39P, S42G, D56I, S57W, S59V, T65V, V76L, Q109R, S116D, T127V, S144P, A147H, S167L and G175D; and a hexosaminidase having SEQ ID NO: 10, 11, 19, 20, 21 or 22.

In some embodiments, the wash liquor or the cleaning composition comprises a protease having SEQ ID NO: 6, 7 or 8 and a DNase having SEQ ID NO: 3 or 4.

Suitable commercially available protease enzymes may include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze® Pro, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Preferenz® P300, Purafect Prime, Preferenz P110™, Effectenz P1000™, Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

In one aspect, the variants of the enzymes (protease, DNase and hexosaminidase) according to the present invention comprise a substitution, deletion, and/or insertion at one or more positions. In one embodiment, the number of amino acid substitutions, deletions and/or insertions introduced into the mature polypeptide of e.g. SEQ ID NO: 2, 3, 5, 6, 10, or 11 is no more than 20, e.g., 1-15, such as 1, 2, 3, 4, 5, 6, 7, 8, 9 or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R.L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for proteas activity, DNase activity or hexosaminidase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

### Additional Enzymes

One or more additional enzymes such as amylases, lipases, cellulases, mannanase, hemicellulases, peroxidases, xylanases, phospholipases, esterases, cutinases, keratinases, reductases, oxidases, phenoloxidases, lipoxygenases, ligninases, pullulanases, malanases, β-glucanases, arabinosidases, hyaluronidase, laccases, perhydrolases and peroxidases.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Amylases

Suitable amylases which can be used together with the protease, the DNase and/or the hexosaminidase of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A18I, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R18I, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131|+T165|+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:
E187P+1203Y+G476K;
E187P+1203Y+R458N+T459S+D460T+G476K;
   wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:
N21D+D97N+V128I
   wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R18I, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

### Cellulases

Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500 (B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

A suitable peroxidase is preferably a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporous, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Protease Stabilizers/Inhibitors

The protease of the present invention may be stabilized using compounds that act by temporarily reducing the proteolytic activity (reversible inhibitors).

Thus, the cleaning composition of the invention may also include a protease inhibitor/stabilizer, which is a reversible inhibitor of protease activity, e.g., serine protease activity. Preferably, the protease inhibitor is a (reversible) subtilisin protease inhibitor. In particular, the protease inhibitor may be a peptide aldehyde, boric acid, or a boronic acid; or a derivative of any of these.

### Boronic Acids

The protease inhibitor may be a boronic acid or a derivative thereof; preferably, a phenylboronic acid or a derivative thereof. In an embodiment of the invention, the phenyl boronic acid derivative is of the following formula:

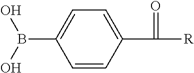

wherein R is selected from the group consisting of hydrogen, hydroxy, C1-C6 alkyl, substituted C1-C6 alkyl, C1-C6 alkenyl and substituted C1-C6 alkenyl. Preferably, R is hydrogen, CH₃, CH₃CH₂ or CH₃CH₂CH₂.

In a preferred embodiment, the protease inhibitor (phenyl boronic acid derivative) is 4-formyl-phenyl boronic acid (4-FPBA).

In another particular embodiment, the protease inhibitor is selected from the group consisting of thiophene-2 boronic acid, thiophene-3 boronic acid, acetamidophenyl boronic acid, benzofuran-2 boronic acid, naphtalene-1 boronic acid, naphtalene-2 boronic acid, 2-FPBA, 3-FBPA, 4-FPBA, 1-thianthrene boronic acid, 4-dibenzofuran boronic acid, 5-methylthiophene-2 boronic, acid, thionaphtrene boronic acid, furan-2 boronic acid, furan-3 boronic acid, 4,4 biphenyl-diborinic acid, 6-hydroxy-2-naphtalene, 4-(methylthio) phenyl boronic acid, 4 (trimethyl-silyl)phenyl boronic acid, 3-bromothiophene boronic acid, 4-methylthiophene boronic acid, 2-naphtyl boronic acid, 5-bromothiphene boronic acid, 5-chlorothiophene boronic acid, dimethylthiophene boronic acid, 2-bromophenyl boronic acid, 3-chlorophenyl boronic acid, 3-methoxy-2-thiophene, p-methyl-phenylethyl boronic acid, 2-thianthrene boronic acid, di-benzothiophene boronic acid, 4-carboxyphenyl boronic acid, 9-anthryl boronic acid, 3,5 dichlorophenyl boronic, acid, diphenyl boronic acidanhydride, o-chlorophenyl boronic acid, p-chlorophenyl boronic acid, m-bromophenyl boronic acid, p-bromophenyl boronic acid, p-flourophenyl boronic acid, p-tolyl boronic acid, o-tolyl boronic acid, octyl boronic acid, 1,3,5 trimethylphenyl boronic acid, 3-chloro-4-flourophenyl boronic acid, 3-aminophenyl boronic acid, 3,5-bis-(triflouromethyl) phenyl boronic acid, 2,4 dichlorophenyl boronic acid, and 4-methoxyphenyl boronic acid.

Further boronic acid derivatives suitable as protease inhibitors in the cleaning composition are described in U.S. Pat. Nos. 4,963,655, 5,159,060, WO 95/12655, WO 95/29223, WO 92/19707, WO 94/04653, WO 94/04654, U.S. Pat. Nos. 5,442,100, 5,488,157 and 5,472,628.

### Peptide Aldehyde or Ketone

The protease stabilizer may have the formula: P-A-L-B-B0-R* wherein:
R* is H (hydrogen), CH₃, CX₃, CHX₂, or CH₂X, wherein X is a halogen atom, particularly F (fluorine); preferably, R*═H (so the stabilizer is a peptide aldehyde with the formula P-A-L-B-B0-H);
L is either absent or a linker group of the formula —C(═O)—, —C(═O)—C(═O)—, —C(═S)—, —C(═S)—C(═S)—, or —C(═S)—C(═O)—;
A is absent if L is absent, or is 1 or 2 amino acid residues connected to L via the N-terminal; thus, A may represent A1 or A2-A1, where A2 and A1 each represent one amino acid residue;
B may be 1, 2, or 3 amino acid residues; thus, B may represent B1, B2-B1, or B3-B2-B1, which is connected to B0 via the C-terminal, where B3, B2, and B1 each represent one amino acid residue; B0 is a single amino acid residue with L- or D-configuration of the formula —NH—CH(R)—C(═O)—;
R is independently selected from the group consisting of C₁₋₆ alkyl, C₆₋₁₀ aryl or C₇₋₁₀ arylalkyl, optionally substituted with one or more, identical or different, substituents R′;
R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N) NH₂;
R″ is a C₁₋₆ alkyl group; and
P is selected from the group consisting of hydrogen, or—if L is absent—an N-terminal protection group;
B0 may be a single amino acid residue with L- or D-configuration, which is connected to H via the C-terminal of the amino acid. B0 has the formula —NH—CH(R)—C(═O)—, wherein R is a C₁-6 alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituents R′. Particular examples of B0 are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B0 is leucine, methionine, phenylalanine, p-tyrosine, or valine. Paticularly preferred is p-tyrosine.

B1, which is connected to B0 via the C-terminal of the amino acid, may be an aliphatic, hydrophobic and/or neutral amino acid. Examples of B1 are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B1 are alanine, glycine, isoleucine, leucine and valine. A particular embodiment is when B1 is alanine, glycine, or valine.

B2, if present, is connected to B1 via the C-terminal of the amino acid, and may be an aliphatic, hydrophobic, neutral and/or polar amino acid. Examples of B2 are alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val). Particular examples of B2 are alanine, arginine, capreomycidine, glycine, isoleucine, leucine, phenylalanine and valine. A particular embodiment is when B2 is arginine, glycine, leucine, phenylalanine, or valine.

B3, if present, is connected to B2 via the C-terminal of the amino acid, and may be a large, aliphatic, aromatic, hydrophobic and/or neutral amino acid. Examples of B3 are isoleucine (Ile), leucine (Leu), norleucine (Nle), norvaline (Nva), phenylalanine (Phe), phenylglycine, tyrosine (Tyr), tryptophan (Trp) and valine (Val). Particular examples of B3 are leucine, phenylalanine, tyrosine, and tryptophan.

The linker group L may be absent or selected from the group consisting of-C(═O)—, —C(═O)—C(═O)—, —C(═S)—, —C(═S)—C(═S)— or —C(═S)—C(═O)—. Particular embodiments of the invention are when L is absent or L is a carbonyl group-C(═O)—.

A1, if present, is connected to L via the N-terminal of the amino acid, and may be an aliphatic, aromatic, hydrophobic, neutral and/or polar amino acid. Examples of A1 are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A1 are alanine, arginine, glycine, leucine, phenylalanine, tyrosine, tryptophan and valine. A particular embodiment is when B2 is leucine, phenylalanine, tyrosine or tryptophan.

A2, if present, is connected to A1 via the N-terminal of the amino acid, and may be a large, aliphatic, aromatic, hydrophobic and/or neutral amino acid. Examples of A2 are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A2 are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy) carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP (OH) (═O)) and benzylphosphoramidyl (PhCH₂OP(OH) (═O)).

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153. More particularly, the peptide aldehyde may be Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, Moc-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH) (O)-Val-Ala-Leu-H, EtSO₂-Phe-Gly-Ala-Leu-H, PhCH₂SO₂-Val-Ala-Leu-H, PhCH₂O—P(OH) (O)-Leu-Ala-Leu-H, PhCH₂O—P(OH) (O)-Phe-Ala-Leu-H, or MeO—P(OH) (O)-Leu-Gly-Ala-Leu-H. A preferred stabilizer for use in the liquid composition of the invention is Cbz-Gly-Ala-Tyr-H, or a hydrosulfite adduct thereof, wherein Cbz is benzyloxycarbonyl.

Further examples of such peptide aldehydes include α-MAPI, β-MAPI, Phe-C(═O)—Arg-Val-Tyr-H, Phe-C(═O)-Gly-Gly-Tyr-H, Phe-C(═O)-Gly-Ala-Phe-H, Phe-C(═O)-Gly-Ala-Tyr-H, Phe-C(═O)-Gly-Ala-L-H, Phe-C(═O)-Gly-Ala-Nva-H, Phe-C(═O)-Gly-Ala-Nle-H, Tyr-C(═O)—Arg-Val-Tyr-H, Tyr-C(═O)-Gly-Ala-Tyr-H, Phe-C(═S)—Arg-Val-Phe-H, Phe-C(═S)—Arg-Val-Tyr-H, Phe-C(═S)-Gly-Ala-Tyr-H, Antipain, GE20372A, GE20372B, Chymostatin A, Chymostatin B, and Chymostatin C.

The protease stabilizer may be a hydrosulfite adduct of the peptide aldehyde or ketone described above, e.g., as described in WO 2013/004636. The adduct may have the formula P-A-L-B—N(H)—CHR—CH(OH)—SO₃M, wherein P, A, L, B, and R are defined as above, and M is H or an alkali metal, preferably Na or K.

An aqueous solution of the hydrosulfite adduct may be prepared by reacting the corresponding peptide aldehyde with an aqueous solution of sodium bisulfite (sodium hydrogen sulfite, NaHSO₃); potassium bisulfite (KHSO₃) by known methods, e.g., as described in WO 98/47523; U.S. Pat. Nos. 6,500,802; 5,436,229; J. Am. Chem. Soc. (1978) 100, 1228; Org. Synth., Coll. vol. 7:361.

Particularly preferred peptide aldehyde protease stabilizers have the formula P-B3-B2-B1-B0-H, or a hydrosulfite adduct having the formula P-B3-B2-B1-N(H)—CHR—CHOH—SO₃M, wherein
i) H is hydrogen;
ii) B0 is a single amino acid residue with L- or D-configuration of the formula —NH—CH(R)—C(═O)—;
iii) B1 and B2 are independently single amino acid residues;
iv) B3 is a single amino acid residue, or is absent;
v) R is independently selected from the group consisting of C₁₋₆ alkyl, C₆₋₁₀ aryl or C₇₋₁₀ arylalkyl optionally substituted with one or more, identical or different, substituents R′;
vi) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N) NH₂;
vii) R″ is a C₁₋₆ alkyl group;
viii) P is an N-terminal protection group, preferably methoxycarbonyl (Moc) or benzyloxycarbonyl (Cbz); and
ix) M is H or an alkali metal, preferably Na or K.

In an even more preferred embodiment, the peptide aldehyde protease stabilizer has the formula P-B2-B1-B0-H or an adduct having the formula P-B2-B1-N(H)—CHR—CHOH—SO₃M, wherein
i) H is hydrogen;
ii) B0 is a single amino acid residue with L- or D-configuration of the formula —NH—CH(R)—C(═O)—;
iii) B1 and B2 are independently single amino acid residues;
iv) R is independently selected from the group consisting of C₁₋₆ alkyl, C₆₋₁₀ aryl or C₇₋₁₀ arylalkyl optionally substituted with one or more, identical or different, substituents R′;
v) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N) NH₂;
vi) R″ is a C₁₋₆ alkyl group;
vii) P is an N-terminal protection group, preferably methoxycarbonyl (Moc) or benzyloxycarbonyl (Cbz); and
viii) M is H or an alkali metal, preferably Na or K.

Preferred embodiments of B0, B1, B2, B3, and P are as described above.

When the peptide aldehyde has the formula P-B3-B2-B1-B0-H, or a hydrosulfite adduct thereof, P is preferably acetyl, methoxycarbonyl, benzyloxycarbonyl, methylamino carbonyl, methylsulfonyl, benzylsulfonyl and benzylphosphoramidyl.

When the peptide aldehyde has the formula P-B2-B1-B0-H, or a hydrosulfite adduct thereof, P is preferably acetyl, methoxycarbonyl, methylsulfonyl, ethylsulfonyl and methylphosphoramidyl.

The molar ratio of the above-mentioned peptide aldehydes (or hydrosulfite adducts) to the protease may be at least 1:1 or 1.5:1, and it may be less than 1000:1, more preferred less than 500:1, even more preferred from 100:1 to 2:1 or from 20:1 to 2:1, or most preferred, the molar ratio is from 10:1 to 2:1.

Formate salts (e.g., sodium formate) and formic acid have also shown good effects as inhibitor of protease activity. Formate can be used synergistically with the above-mentioned protease inhibitors, as shown in WO 2013/004635. The formate salts may be present in the composition in an amount of at least 0.1% w/w or 0.5% w/w, e.g., at least 1.0%, at least 1.2% or at least 1.5%. The amount is typically below 5% w/w, below 4% or below 3%.

In an embodiment, the protease is a metalloprotease and the inhibitor is a metalloprotease inhibitor, e.g., a protein hydrolysate based inhibitor (e.g., as described in WO 2008/134343).

### Surfactants

The cleaning composition of the invention may comprise one or more surfactants, which may be anionic and/or cationic and/or non-ionic and/or semi-polar and/or zwitterionic, or a mixture thereof. In a particular embodiment, the cleaning composition includes a surfactant system (comprising more than one surfactant) e.g. a mixture of one or more nonionic surfactants and one or more anionic surfactants. In one embodiment the detergent comprises at least one anionic surfactant than at least one non-ionic surfactant, the weight ratio of anionic to nonionic surfactant may be from 10:1 to 1:10. In one embodiment the amount of non-ionic surfactant is higher than the amount of anionic surfactant e.g. the weight ratio of non-ionic to anionic surfactant may be from 10:1 to 1.1:1 or from 5:1 to 1.5:1. The amount of anionic to non-ionic surfactant may also be equal and the weight ratios 1:1. A medical cleaning detergent (e.g., a cleaning composition for cleaning a medical device) usually comprises more nonionic than anionic surfactant. The total weight of surfactant(s) present in the composition is typically from about 0.1% to about 60% by weight, such as about 1% to about 40%, or about 3% to about 20%, or about 3% to about 10%. The surfactants are chosen based on the desired cleaning application, and may include any conventional surfactant(s) known in the art. When included therein the cleaning composition will usually contain from about 1% to about 30% by weight of an anionic surfactant, such as from about 5% to about 20%, or from about 5% to about 10% of an anionic surfactant. Non-limiting examples of anionic surfactants include sulfates and sulfonates, typically available as sodium or potassium salts or salts of monoethanolamine (MEA, 2-aminoethan-1-ol) or triethanolamine (TEA, 2,2′,2″-nitrilotriethan-1-ol); in particular, linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids. Furthermore, salts of fatty acids (soaps) may be included.

When included therein the cleaning composition will usually contain from about 1% to about 30% by weight of a cationic surfactant, for example from about 0.5% to about 20%, in particular from about 1% to about 15%, from about 3% to about 10%, such as from about 3% to about 5. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the cleaning composition will usually contain from about 1% to about 40% by weight of a nonionic surfactant, for example from about 5% to about 30%, in particular from about 2% to about 20%, from about 3% to about 10%, such as from about 5% to about 25%, from about 8% to about 15. Non-limiting examples of nonionic surfactants include alcohol ethoxylates (AE or AEO) e.g. the AEO-series such as AEO-7, alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the cleaning composition will usually contain from about 0.01 to about 10% by weight of a semipolar surfactant. Non-limiting examples of semipolar surfactants include amine oxides (AO) such as alkyldimethylamine oxides, in particular N-(coco alkyl)-N,N-dimethylamine oxide and N-(tallow-alkyl)-N, N-bis(2-hydroxyethyl)amine oxide, and combinations thereof.

When included therein the cleaning composition will usually contain from about 0.01% to about 10% by weight of a zwitterionic surfactant. Non-limiting examples of zwitterionic surfactants include betaines such as alkyldimethylbetaines, sulfobetaines, and combinations thereof.

Additional bio-based surfactants may be used e.g, wherein the surfactant is a sugar-based non-ionic surfactant which may be a hexyl-β-D-maltopyranoside, thiomaltopyranoside or a cyclic-maltopyranoside, such as described in EP2516606 B1.

### Hydrotropes

A hydrotrope is a compound that solubilizes hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12:121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in cleaning compositions allow for example more concentrated formulations of surfactants (as in the process of compacting liquid detergents by removing water) without inducing undesired phenomena such as phase separation or high viscosity.

The cleaning composition may contain 0-10% by weight, for example 0.2-8% by weight, such as about 0.5 to about 5%, or about 3% to about 5%, of a hydrotrope. Any hydrotrope known in the art for use in a cleaning composition may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The cleaning composition may contain about 0-50% by weight, such as about 0.5% to about 30%, 1-10% of a detergent builder or co-builder, or a mixture thereof. The builder and/or co-builder may particularly be a chelating agent that forms water-soluble complexes with Ca and Mg. Any builder and/or co-builder known in the art for use in cleaning detergents may be utilized.

Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Clariant), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl) inulin (CMI), and combinations thereof.

The cleaning composition may also contain from about 0-50% by weight, such as about 5% to about 30%, of a detergent co-builder. The cleaning composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid (HEDP), ethylenediaminetetramethylenetetrakis (phosphonic acid) (EDTMPA), diethylenetriaminepentamethylenepentakis (phosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), aminotrimethylenetris(phosphonic acid) (ATMP), and combinations and salts thereof. Further exemplary builders and/or co-builders are described in, e.g., WO 09/102854, U.S. Pat. No. 5,977,053.

### Polymers

The cleaning composition may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties. Exemplary polymers include (carboxymethyl) cellulose (CMC), poly(vinyl alcohol) (PVA), poly(ethyleneglycol) or poly(ethylene oxide) (PEG or PEO), ethoxylated poly(ethyleneimine), (carboxymethyl) inulin (CMI), carboxylate polymers and d, and lauryl methacrylate/acrylic acid copolymers, hydrophobically modified CMC (HM-CMC), silicones, copolycarboxylates such as polyacrylates, maleic/acrylic acid copolymers, acrylate/styrene copolymers, poly(aspartic) acipolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Particularly preferred polymer is ethoxylated homopolymer Sokalan® HP 20 from BASF, which helps to prevent redeposition of soil in the wash liquor. Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

### Adjunct Materials

Any detergent adjunct components known in the art may also be utilized. Exemplary adjunct materials may include anti-corrosion agents, anti-shrink agents, anti-soil redeposition agents, bactericides, corrosion inhibitors (or “rust inhibitor”), disintegrates/disintegration agents, dyes, enzyme stabilizers (including boric acid, borates, CMC, and/or polyols such as propylene glycol), suds suppressors, either alone or in combination. The choice of such ingredients is well within the skill of the artisan. In some embodiments, the cleaning compositions of the present invention may include a corrosion inhibitor to prevent rust or corrosion of the medical devices after washing or cleaning.

### Formulation of Cleaning Composition Products

The cleaning composition of the invention may be formulated in any convenient form, e.g., a liquid, a tablet, a pouch having one or more compartments, a foam or a spray. In one embodiment, the cleaning composition is formulated as a liquid type detergent.

Pouches can be configured as single or multicompartments. They can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid cleaning composition or part components and/or a liquid cleaning composition or part components separated by the water soluble film.

Detergent ingredients or cleaning components can be separated physically from each other by compartments in water dissolvable pouches or in different layers of tablets. Thereby negative storage interaction between components can be avoided.

A liquid or gel detergent which is not unit dosed may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Formulation of Enzyme in Co-Granule

The enzymes of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates is disclosed in WO 2013/188331, which relates to a cleaning composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

The multi-enzyme co-granule may comprise enzymes of the invention and one or more additional enzymes selected from the group consisting of amylases, lipases, cellulases, mannanases, hemicellulases, peroxidases, xylanases, phospholipases, esterases, cutinases, keratinases, reductases, oxidases, phenoloxidases, lipoxygenases, ligninases, pullulanases, malanases, ß-glucanases, arabinosidases, hyaluronidase, laccases, perhydrolases, peroxidases and mixtures thereof.

The present invention is further described in the following paragraphs:

1. A method of cleaning a medical device, comprising the steps of:
(a) contacting the medical device with a wash liquor comprising two or more enzymes selected from the group consisting of a protease, an enzyme having DNase activity and an enzyme having hexosaminidase activity; and optionally
(b) rinsing the medical device.

2. Method according to paragraph 1, wherein the medical device is or comprises an endoscope such as a cystoscope, a nephroscope, a bronchoscope, a laryngoscope, an otoscope, an arthroscope, a laparoscope, and a gastrointestinal endoscope; a surgical instrument such as a scalpel, a hemostat, a forceps, scissors, a retractor, a tracheotome and a clamp; and dental instruments e.g. scalers, curettes, serrated cotton pliers, dental mirrors.

3. Method according to paragraph 1 or 2, wherein the method further comprises a step of pre-treating the medical device with a disinfectant before step (a), and preferably the disinfectant is selected from the group consists of peracetic acid, hydrogen peroxide, potassium permanganate, chlorine dioxide and ethanol.

4. Method according to any of the preceding paragraphs, wherein the medical device is rinsed with water or with a solution comprising a disinfectant such as peracetic acid, hydrogen peroxide, potassium permanganate, chlorine dioxide or ethanol.

5. Method according to any of proceeding paragraphs, wherein the medical device is soiled with biofilm, wherein the biofilm is produced by or partly produced by Escherichia coli, Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538, Streptococcus (e.g., S. pyogenes, S. agalacticae or S. pneumoniae), Haemophilus influenzae, Pseudomonas aeruginosa, Clostridium perfringens, Chlamydia trachomatis, Candida albicans, and or Bacillus anthracis.

6. Method according to any of the preceding paragraphs, wherein the enzyme having DNase activity is of microbial origin, e.g. fungal or bacterial origin.

7. Method according to any of the preceding paragraphs, wherein the enzyme having DNase activity is a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 1, 2, 3 or 4.

8. Method according to any of the preceding paragraphs, wherein the enzyme having DNase activity comprises one or more (e.g., two, three, four, five or six) of the motifs selected from the group consisting of [T/D/S][G/N]PQL (SEQ ID NO: 23), [F/L/Y/I]A[N/R]D[L/I/P/V] (SEQ ID NO: 24), C[D/N]T[A/R] (SEQ ID NO: 25), [D/Q][I/V]DH (SEQ ID NO: 26), [D/M/L][S/T]GYSR[D/N] (SEQ ID NO: 27) and ASXNRSKG (SEQ ID NO: 28), and has at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4.

9. Method according to any of the preceding paragraphs, wherein the enzyme having DNase activity comprises one or both motifs [D/M/L][S/T]GYSR[D/N] (SEQ ID NO: 27) and ASXNRSKG (SEQ ID NO: 28), and optionally further comprises one or more (e.g., two, three, four) of the motifs selected from the group consisting of [T/D/S][G/N]PQL (SEQ ID NO: 23), [F/L/Y/I]A[N/R]D[L/I/P/V] (SEQ ID NO: 24), C[D/N]T[A/R] (SEQ ID NO: 25), [D/Q][I/V]DH (SEQ ID NO: 26), and wherein the enzyme having DNase activity has at 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4.

10. Method according to any of the preceding paragraphs, wherein the enzyme having DNase activity is a variant of SEQ ID NO: 2, comprising one or more alterations selected from the group consisting of S26*, D32E,Q, V35I, K36C,H, G37R, F43W, D46G, A55I, N68D, S69V, A76I, K82S, T, P84D,T, K86E,G,L,N,Q,T,V,Y, A91R, L92E, K95I, P97E,N, A101E, Q102E, K105N,G,Q,T,D, A111P, F112Y,W, S115T, V127T, L129K, N133Q, G137R, V138C, N140H, G141Q,R, S144E, N146A, K147N,E, V148I, A149D,E,F, Q150D,E, P153D,V, S154E, K155E,F,L,S,T, Q157D,E, Q158D, T159Q, K160D, G161R, T170Q, A172D,E,H,R, G181N, K185*, V187N, Y, N191*, K192A,I, D197K,S, G199Q, Q208V, E211Y,T,P, N213S, N214D, N217A and Y218D, E, wherein position numbers correspond to the positions of SEQ ID NO: 2, wherein the variant has DNase activity and has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

11. Method according to any of the preceding paragraphs, wherein the enzyme having DNase activity is a variant comprising one or more alterations at positions 1, 13, 22, 25, 27, 33, 39, 42, 56, 57, 59, 65, 76, 77, 109, 116, 127, 144, 147, 149, 167, 175 and 181 of SEQ ID NO: 4, wherein the variant has DNase activity and has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 4.

12. Method according to paragraph 9, wherein the enzyme having DNase activity is a variant of SEQ ID NO: 4, comprising one or more substitutions of T1I, S13Y, T22P, S25P, S27L, L33K, S39P, S42G, D56I, S57W, S59V, T65V, V76L, T77Y, Q109R, S116D, T127V, S144P, A147H, G149N, S167L, G175D, and S181L, e.g., comprising T22P+S167L and/or G175D, wherein position numbers correspond to the positions of SEQ ID NO: 4, and wherein the variant has DNase activity and has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 4, and preferably wherein the variant comprises or consists of SEQ ID NO: 4 with the substitutions T1I, S13Y, T22P, S27L, L33K, S39P, S42G, D56I, S57W, S59V, T65V, V76L, Q109R, S116D, T127V, S144P, A147H, S167L and G175D.

13. Method according to any of the preceding paragraphs, wherein the enzyme having DNase activity is a variant of SEQ ID NO: 4 with the following substitutions: T1l+S13Y+T22P+S27L+L33K+S39P+S42G+D56I+S57W+S59V+T65V+V76L+Q109R+S116D+T127V+S144P+A147H+S167L+G175D.

14. Method according to any of the preceding paragraphs, wherein the enzyme having DNase activity is a variant of SEQ ID NO: 4 with the following substitutions: T1I+S13Y+T22P+S25P+S27L+S39P+S42G+S57W+S59V+T65V+V76L+T77Y+Q109R+S116D+S144P+A147H+G149N+S167L+G175D+S181L.

15. Method according to any of the preceding paragraphs, wherein the enzyme having DNase activity is a variant of SEQ ID NO: 2 with the following substitutions: S69V+Q102E+K105N+A111P+S115T+Q150E+G161R+G181N+V187N+K192I.

16. Method according to any of the preceding paragraphs, wherein the enzyme having DNase activity is a variant of SEQ ID NO: 2 with the following substitutions: D32E+V35I+S69V+K86E+Q102E+K105N+A111P+S115T+V127T+G137R+K147N+Q150E+K155E+T159Q+G161R+A172D+G181N+V187N+K192A+Q208V+N217A.

17. Method according to any of the preceding paragraphs, wherein the enzyme having hexosaminidase activity is of microbial origin, e.g. fungal or bacterial origin.

18. Method according to any of the preceding paragraphs, wherein the enzyme having hexosaminidase activity has N-acetylglucosaminidase activity and/or β-N-acetylglucosamininidase activity.

19. Method according to any of the preceding paragraphs, wherein the enzyme having hexosaminidase activity is a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10, 11, 19, 20, 21 or 22.

20. Method according to any of the preceding paragraphs, wherein the enzyme having hexosaminidase activity is a variant of polypeptide of SEQ ID NO:10, comprising one or more alterations at positions 3, 15, 49, 59, 163, 186, 225, 227, 232, 235, 252, 260, 272, 279, 281, 308, 309 and 312 of SEQ ID NO: 10, wherein the variant has hexosaminidase activity and has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 10.

21. Method according to paragraph 18, wherein the enzyme having hexosaminidase activity is a variant of polypeptide of SEQ ID NO:10, comprising one or more substitutions of Q3I, H15Y, A49W, N59E, S163P, S186R, S225G, N227T, E232D, G235W, N252P, N260Q, H272V, S279D, Y281P, K308Q, K309E and K312Q of SEQ ID NO: 10, wherein position numbers correspond to the positions of SEQ ID NO: 10, and wherein the variant has hexosaminidase activity and has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 10, and preferably wherein the variant comprises or consists of SEQ ID NO: 10 with the substitutions Q3I, H15Y, A49W, N59E, S163P, S186R, S225G, N227T, E232D, G235W, N252P, N260Q, H272V, S279D, Y281P, K308Q, K309E and K312Q.

22. Method according to any of the preceding paragraphs, wherein the polypeptide having hexosaminidase activity comprises one or more of the motifs selected from the group consisting of GXDE (SEQ ID NO: 12), [EQ][NRSHA][YVFL][AGSTC][IVLF][EAQYN][SN] (SEQ ID NO: 13), [VIM][LIV]G[GAV]DE[VI][PSA] (SEQ ID NO: 14), WND [SQR][IVL][TLVM] (SEQ ID NO: 15), QSTL (SEQ ID NO: 16), NKFFY (SEQ ID NO: 17) and NLD[DR]S (SEQ ID NO: 18).

23. Method according to any of the preceding paragraphs, wherein the protease has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 5, 6, 7, 8, 9 or 29.

24. Method according to paragraph 20, wherein the protease is a variant of the polypeptide of SEQ ID NO: 6 comprising an alteration at one or more positions corresponding to positions 3, 4, 9, 15, 22, 43, 68, 76, 87, 99, 101, 103, 104, 118, 128, 160, 167, 170, 184, 194, 199, 205, 206, 209, 217, 218, 222, 245, 259, 261 and 262, wherein position numbers correspond to the positions of SEQ ID NO: 5, wherein each alteration is independently a substitution, deletion or insertion, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 6.

25. Method according to any of the proceeding paragraphs, wherein the protease is:
(a) a variant of the polypeptide of SEQ ID NO: 6, comprising one or more substitutions selected from the group consisting of: S3T, V4I, S9E, S9R, A15T, T22A, N43R, V68A, N76D, S87N, S99D, S99G, S99A, S99SE, S101E, S101N, S101R, S103A, V104I, G118M, S128Q, G160S, Y167A, R170S, N184E, A194P, V199M, V205I, Q206L, Y209W, L217D, L217Q, N218D, M222S, Q245R, S259D, N261W and L262E, e.g., comprising substitutions of N76D+Q206L+Y209W or Y209W+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, at least 99%, but less than 100% sequence identity to SEQ ID NO: 6;
(b) a variant of the polypeptide of SEQ ID NO: 6, comprising the substitution S87N, wherein the variant has protease activity and wherein the position corresponds to the position of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(c) a variant of the polypeptide of SEQ ID NO: 6, comprising the substitutions Y167A+R170S+A194P, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(d) a variant of the polypeptide of SEQ ID NO: 6, comprising the substitutions S9E+N43R+N76D+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(e) a variant of the polypeptide of SEQ ID NO: 6, comprising the substitutions S3T+N43R+N76D+S87N+G118M+S128Q+N184E+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(f) a variant of the polypeptide of SEQ ID NO: 6, comprising the substitutions T22A+N43R+S87N+V205L+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(g) a variant of the polypeptide of SEQ ID NO: 8, comprising the substitutions A68S+T77N+T78I+G127S+A128P+G165Q+N184Q+A202V+N217S+S258P, wherein position numbers correspond to the positions of SEQ ID NO: 8, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO:8;
(h) a variant comprising a substitution at one or more positions corresponding to positions 171, 173, 175, 179 or 180 of SEQ ID NO: 1 of WO2004/067737, wherein the variant has protease activity and has a sequence identity of at least 80%, at least 85%, at least 90%, at least 91%, at least 92%, at least 93%, at least 94%, at least 95%, at least 96%, at least 97%, at least 98%, or at least 99%, but less than 100% to SEQ ID NO: 1 of WO2004/067737;
(i) a protease variant comprising one or more substitutions compared to a parent protease, selected from the group consisting of X3V, X9 [E,R], X22 [R,A], X43R, X61 [E,D], X62 [E,D], X76 [D], X87N, X101 [E,G,D,N,M], X103A, X104I, X118 [V,R,M], X120V, X128 [A,L,S,Q], X129Q, X130A, X160D, X184 [E,D], X185 [E,D], 188 [E,D], X191N, X194P, X205I, X206L, X209W, X216V, X217 [Q,D,E], X218 [D,E,S], X232V, X245R, X248D, X256 [E,D], X259 [E,D], X261 [E,D,W] and X262 [E,D], wherein position numbers correspond to the positions of BPN′ (SEQ ID NO: 5), wherein “X” represents any amino acid residue present in the specified position in the parent protease, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or SEQ ID NO: 6; and
(j) a protease variant comprising any of the following substitution sets compared to a parent protease, wherein the parent protease has the amino acid sequence of SEQ ID NO: 5 or SEQ ID NO: 6 or has at least 80% sequence identity to SEQ ID NO: 5 or SEQ ID NO: 6, wherein position numbers correspond to the positions of BPN′ (SEQ ID NO: 5), wherein “X” represents any amino acid residue present in the specified position in the parent protease, and wherein the substitution set is selected from the group consisting of:
   i. X9R+X15T+X68A+X218D+X245R,
   ii. X9R+X15T+X68A+X245R,
   iii. X61E+X194P+X205I+X261D,
   iv. X61D+X205I+X245R,
   v. X61E+X194P+X205I+X261D,
   vi. X87N+X118V+X128L+X129Q+X130A,
   vii. X87N+X101M+X118V+X128L+X129Q+X130A,
   viii. X76D+X87R+X118R+X128L+X129Q+X130A,
   ix. X22A+X62D+X101G+X188D+X232V+X245R,
   x. X103A+X104I,
   xi. X22R+X101G+X232V+X245R,
   xii. X103A+X104I+X156D,
   xiii. X103A+X104I+X261E,
   xiv. X62D+X245R,
   xv. X101N+X128A+X217Q,
   xvi. X101E+X217Q,
   xvii. X101E+X217D,
   xviii. X9E+X43R+X262E,
   xix. X76D+X43R+X209W,
   xx. X205I+X206L+X209W,
   xxi. X185E+X188E+X205I,
   xxii. X256D+X261W+X262E,
   xxiii. X191N+X209W,
   xxiv. X261E+X262E,
   xxv. X261E+X262D, and
   xxvi. X167A+X170S+X194P,
wherein the protease variant has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or 6.

26. Method according to any of the preceding paragraphs, wherein the protease variant comprises the amino acid sequence of SEQ ID NO: 6 with the substitutions Y167A+R170S+A194P, wherein the variant has protease activity and wherein position numbers correspond to the positions of SEQ ID NO: 5.

27. Method according to any of the preceding paragraphs, wherein the protease variant comprises the amino acid sequence of SEQ ID NO: 6 with the substitutions S9E+N43R+N76D+V205I+Q206L+Y209W+S259D+N261W+L262E, wherein the variant has protease activity and wherein position numbers correspond to the positions of SEQ ID NO: 5.

28. Method according to any of the preceding paragraphs, wherein the protease is an enzyme having or consisting of the amino acid sequence of SEQ ID NO: 6, 7, 8 or 9.

29. Method according to any of the preceding paragraphs, wherein the amount of biofilm existing on the medical device after cleaning is reduced compared to cleaning with a wash liquor without said two or more enzymes.

30. Method according to any of the preceding paragraphs, wherein the wash liquor further comprises one or more additional enzymes selected from the group consisting of amylases, lipases, cellulases, mannanases, hemicellulases, peroxidases, xylanases, phospholipases, esterases, cutinases, keratinases, reductases, oxidases, phenoloxidases, lipoxygenases, ligninases, pullulanases, malanases, β-glucanases, arabinosidases, hyaluronidase, laccases, perhydrolases and peroxidases.

31. Method according to any of the preceding paragraphs, wherein the concentration of each enzyme in the wash liquor is in the range of 0.0005-100 ppm enzyme protein, such as in the range of 0.001-50 ppm, in the range of 0.005-20 ppm, in the range of 0.01-10 ppm, or in the range of 0.05-5 ppm enzyme protein.

32. Method according to any of the preceding paragraphs, wherein the wash liquor comprises the cleaning composition according to any of paragraphs 37-50.

33. Use of an enzyme composition for cleaning a medical device, wherein the enzyme composition comprises two or more enzymes selected from the group consisting of a protease, an enzyme having DNase activity and an enzyme having hexosaminidase activity.

34. Use according to the preceding use paragraph, wherein the medical device comprises an endoscope such as a cystoscope, a nephroscope, a bronchoscope, a laryngoscope, an otoscope, an arthroscope, a laparoscope, and a gastrointestinal endoscope; a surgical instrument such as a scalpel, a hemostat, a forceps, scissors, a retractor, a tracheotome and a clamp; and dental instruments e.g. scalers, curettes, serrated cotton pliers, dental mirrors.

35. Use according to any of the proceeding use paragraphs, wherein the medical device is soiled with biofilm, wherein the biofilm is produced by or partly produced by Enterobacteriacae (e.g., Escherichia coli), Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538, Streptococcus (e.g., S. pyogenes, S. agalacticae or S. pneumoniae), Haemophilus influenzae, Pseudomonas aeruginosa, Clostridium perfringens, Chlamydia trachomatis, Candida albicans, and/or Bacillus anthracis.

36. Use according to any of the preceding use paragraphs, wherein the enzyme having DNase activity is as defined in any of preceding paragraphs 6-16.

37. Use according to any of the preceding use paragraphs, wherein the enzyme having hexosaminidase activity is as defined in any of preceding paragraphs 17-22.

38. Use according to any of the preceding use paragraphs, wherein the protease is as defined in any of preceding paragraphs 23-28.

39. Use according to any of the preceding use paragraphs, wherein the amount of biofilm existing on the medical device after cleaning is reduced compared to cleaning with a composition without said two or more enzymes.

40. Use according to any of the preceding use paragraphs, wherein a composition according to paragraph 41-54 is used.

41. A composition for cleaning a medical device, comprising a surfactant and two or more enzymes selected from the group consisting of a protease, an enzyme having DNase activity and an enzyme having hexosaminidase activity.

42. Composition according to paragraph 39, wherein the medical device is soiled with biofilm, wherein the biofilm is produced by or partly produced by Enterobacteriacae (e.g., Escherichia coli), Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538, Streptococcus (e.g., S. pyogenes, S. agalacticae or S. pneumoniae), Haemophilus influenzae, Pseudomonas aeruginosa, Clostridium perfringens, Chlamydia trachomatis, Candida albicans, Bacillus anthracis.

43. Composition according to any of the preceding composition paragraphs, wherein the enzyme having DNase activity is as defined in any of preceding paragraphs 6-16.

44. Composition according to any of the preceding composition paragraphs, wherein the enzyme having hexosaminidase activity is as defined in any of preceding paragraphs 17-22.

45. Composition according to any of the preceding composition paragraphs, wherein the protease is as defined in any of preceding paragraphs 23-28.

46. Composition according to any of the preceding composition paragraphs, wherein the amount of biofilm existing on the medical device after cleaning with said composition is reduced compared to cleaning with a composition without said two or more enzymes.

47. Composition according to any of the proceeding composition paragraphs, wherein the surfactant comprises at least a non-ionic surfactant, and optionally further comprises a cationic surfactant and/or an anionic surfactant.

48. Composition according to paragraph 45, wherein the non-ionic surfactant is selected from the group consisting of alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides and N-acyl N-alkyl derivatives of glucosamine (glucamides, GA, or fatty acid glucamides, FAGA).

49. Composition according to paragraph 45, wherein the anionic surfactant is selected from the group consisting of linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, secondary alkanesulfonates (SAS), paraffin sulfonates (PS), ester sulfonates, sulfonated fatty acid glycerol esters, alpha-sulfo fatty acid methyl esters (alpha-SFMe or SES) methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap).

50. Composition according to paragraph 45, wherein the cationic surfactant is selected from the group consisting of bis(Acyloxyethyl) hydroxyethyl Methylammonium Methosulphate, Dipalmoylethyl hydroxyethylmonium methosulfate, dihydrogenated tallow hydroxyethylmonium methosulfate, distearoylethyl hydroxyethylmonium methosulfate, dioleoyl ethyl hydroxyethylmonium methosulfate alkyl quaternary ammonium compounds, alkoxylated quaternary ammonium (AQA) compounds, other ester quats, and combinations thereof.

51. Composition according to any of the preceding composition paragraphs, wherein the composition comprises at least 1 wt %, such as at least 2 wt %, at least 5 wt % of nonionic surfactant, and optionally further comprises 0.2-10 wt % of builder.

52. Composition according to any of the preceding composition paragraphs, wherein the composition further comprises one or more additional enzymes selected from the group consisting of amylases, lipases, cellulases, mannanase, hemicellulases, peroxidases, xylanases, phospholipases, esterases, cutinases, keratinases, reductases, oxidases, phenoloxidases, lipoxygenases, ligninases, pullulanases, malanases, β-glucanases, arabinosidases, hyaluronidase, laccases, perhydrolases and peroxidases.

53. Composition according to any of the preceding composition paragraphs, wherein the composition is a liquid detergent, a foam detergent or a spray detergent.

54. Composition according to any of the preceding composition paragraphs, wherein the composition comprises at least 0.0001 mg of enzyme protein per gram of the composition, at least 0.001 mg of enzyme protein, at least 0.006 mg of enzyme protein, at least 0.008 mg of enzyme protein, at least 0.01 mg of enzyme protein, at least 0.1 mg of enzyme protein, at least 0.5 mg of enzyme protein, at least 1 mg of enzyme protein, at least 2 mg of enzyme protein, at least 5 mg of enzyme protein, at least 10 mg of enzyme protein, or at least 20 mg of enzyme protein.

55. A CIP (cleaning-in-place) cleaning method, comprising performing a cleaning-in-place process using two or more enzymes selected from the group consisting of a protease, an enzyme having DNase activity and an enzyme having hexosaminidase activity; and optionally including a rinse step.

The invention is further illustrated in the following non-limiting examples.

### EXAMPLES

### Materials and Methods

### Model Detergent MC (Liquid)

This is an example of a cleaning composition that can be used in combination with the enzymes of the present invention.

Ingredients: 5% MPG (propylene glycol), 5% Pluronic PE 4300 (PO/EO block polymer; 70%/30%, approx. 1750 g/mol), 2% Plurafac LF 305 (fatty alcohol alkoxylate; C6-10+EO/PO), 1% MGDA (methyl glycine diacetic acid, 1% TEA (triethanolamine) (all percentages are w/w). The pH was adjusted to 8.7 with phosphoric acid.

### Enzyme Assays

### Assay I

### Testing of DNase Activity

DNase activity may be determined on DNase Test Agar with Methyl Green (BD, Franklin Lakes, NJ, USA), which is prepared according to the supplier's manual. Briefly, 21 g of agar is dissolved in 500 ml water and then autoclaved for 15 min at 121° C. Autoclaved agar is tempered to 48° C. in a water bath, and 20 ml of agar is poured into petri dishes and allowed to solidify by incubation overnight at room temperature. On solidified agar plates, 5 μl of enzyme solutions are added, and DNase activity is observed as colorless zones around the spotted enzyme solutions.

### Assay II

### Testing of Hexosaminidase Activity

Hexosaminidase activity may be determined using 4-nitrophenyl N-acetyl-β-D-glucosaminide (Sigma-Aldrich) as a substrate. The enzymatic reaction is performed in triplicate in a 96 well flat bottom polystyrene microtiter plate (Thermo Scientific) with the following conditions: 50 mM 2-(N-morpholino) ethanesulfonic acid pH 6 buffer, 1.5 mg/ml 4-nitrophenyl N-acetyl-β-D-glucosaminide and 10, 20 or 50 μg/ml purified enzyme sample in a total reaction volume of 100 μl. Blank samples without polypeptide are run in parallel. The reactions are carried out at 37° C. in a Thermomixer comfort (Eppendorf). After 10 minutes of incubation, 5 μl 1 M NaOH is added to each reaction mixture to stop the enzymatic reaction. The absorbance is read at 405 nm using a POLAR star Omega plate reader (BMG LABTECH) to estimate the formation of 4-nitrophenolate ion released because of enzymatic hydrolysis of the 4-nitrophenyl N-acetyl-β-D-glucosaminide substrate. A measured absorbance of the reaction carried out with a hexosaminidase polypeptide that is higher than that of blanks without a polypeptide indicates that the tested polypeptide exhibits hexosaminidase activity.

### Assay III

### Testing of Protease Activity

Proteolytic activity can be determined by a method employing the Suc-AAPF-pNA substrate. Suc-AAPF-pNA is an abbreviation for N-Succinyl-Alanine-Alanine-Proline-Phenylalanine-p-Nitroanilide, and is a blocked peptide which can be cleaved by endo-proteases. Following proteolytic cleavage, a free pNA molecule having a yellow color is liberated and can be measured by visible spectrophotometry at wavelength 405 nm. The Suc-AAPF-PNA substrate is manufactured by Bachem (cat. no. L1400, dissolved in DMSO).

The protease sample to be analyzed is diluted in residual activity buffer (100 mM Tris pH 8.6). The assay is performed by transferring 30 μl of diluted enzyme samples to 96 well microtiter plate and adding 70 μl substrate working solution (0.72 mg/ml in 100 mM Tris pH 9). The solution is mixed at room temperature and absorption is measured every 20 sec. over 5 minutes at OD 405 nm.

The slope (absorbance per minute) of the time dependent absorption-curve is directly proportional to the activity of the protease in question under the given set of conditions. The protease sample should be diluted to a level where the slope is linear.

### Enzymes

DNase 1: a DNase corresponding to the polypeptide of SEQ ID NO: 3.
DNase 2: a DNase corresponding to the polypeptide of SEQ ID NO: 4.
DNase 3: polypeptide of SEQ ID NO: 4 with the following substitutions: T1I+S13Y+T22P+S27L+L33K+S39P+S42G+D56I+S57W+S59V+T65V+V76L+Q109R+S116D+T127V+S144P+A147H+S167L+G175D.
DNase 4: polypeptide of SEQ ID NO: 2 with the following substitutions: S69V+Q102E+K105N+A111P+S115T+Q150E+G161R+G181N+V187N+K192I.
DNase 5: polypeptide of SEQ ID NO: 2 with the following substitutions: D32E+V35I+S69V+K86E+Q102E+K105N+A111P+S115T+V127T+G137R+K147N+Q150E+K155E+T159Q+G161R+A172D+G181N+V187N+K192A+Q208V+N217A.
DNase 6: polypeptide of SEQ ID NO: 4 with the following substitutions: T1l+S13Y+T22P+S25P+S27L+S39P+S42G+S57W+S59V+T65V+V76L+T77Y+Q109R+S116D+S144P+A147H+G149N+S167L+G175D+S181L.
Protease 1: polypeptide of SEQ ID NO: 6 with the following mutations: Y167A+R170S+A194P, wherein position numbers are based on the numbering of SEQ ID NO: 5.
Protease 2: a protease corresponding to the polypeptide of SEQ ID NO: 8.
Protease 3: a protease corresponding to the polypeptide of SEQ ID NO: 7.
Protease 4: polypeptide of SEQ ID NO: 6 with the following mutations: S9E+N43R+N76D+V205I+Q206L+Y209W+S259D+N261W+L262E, wherein position numbers are based on the numbering of SEQ ID NO: 5.
Hexosaminidase 1: a hexosaminidase corresponding to the polypeptide of SEQ ID NO: 10.
Hexosaminidase 2: polypeptide of SEQ ID NO: 10 with the following mutations: Q31+H15Y+A49W+N59E+S163P+S186R+S225G+N227T+E232D+G235W+N252P+N260Q+H272V+S279D+Y281P+K308Q+K309E+K312Q.
Hexosaminidase 3: a hexosaminidase corresponding to the polypeptide of SEQ ID NO: 11.
Hexosaminidase 4: a hexosaminidase corresponding to the polypeptide of SEQ ID NO: 19.
Hexosaminidase 5: a hexosaminidase corresponding to the polypeptide of SEQ ID NO: 20.
Hexosaminidase 6: a hexosaminidase corresponding to the polypeptide of SEQ ID NO: 21.
Hexosaminidase 7: a hexosaminidase corresponding to the polypeptide of SEQ ID NO: 22.

### Example 1

### Biofilm Removal Test in Medical Cleaning Model Detergent MC

Staphylococcus aureus ATCC 6538 was used as a model microorganism in the present example. The strain was restreaked on TSA plate (15 g/L tryptone, 5 g/L soya peptone, 5 g/L NaCl and 15 g/L agar, pH 7.340.2) and incubated at 30° C. overnight. The strain was then inoculated into 5 mL TSB medium (15 g/L tryptone, 5 g/L soya peptone, 5 g/L NaCl, pH 7.3±0.2) and incubated for 8 hours at 30° C. with shaking at 200 rpm. The culture was subsequently diluted (1:100 v/v) in TSB+0.25 wt % glucose (G7528, Sigma-Aldrich), and 100 UL aliquots were added to the wells of 96-well polystyrene microplates (167008, Nunc™ MicroWell™ 96-Well, Nunclon Delta-Treated, Flat-Bottom Microplate). Sterile medium was added to control wells. The 96-well microtiter plate was then incubated for 20 hours at 30° C. under static conditions. After incubation, the culture was removed and rinsed with deionized water once. Then the bacteria were disinfected by 0.1% peracetic acid before the wash step.

A liquid medical cleaning model detergent MC, with the composition given above was used in this example. The wash liquor (5 g/L model detergent MC in deionized water) containing either no enzyme (i.e., control) or 5 ppm of one or more enzymes (protease, DNase and/or hexosaminidase) was added to the 96-well microtiter plate and the plate was incubated at 40° C. for 20 minutes. The wells were subsequently rinsed with deionized water and stained with 0.1% crystal violet (C₆₁₅₈, Sigma-Aldrich) at room temperature for 5 minutes. The wells were then rinsed in deionized water once. The remaining dye was dissolved with 30% acetic acid and the resulting mixture was subjected to absorbance measurement at 595 nm (A595) with a microplate reader (SpectraMax® M2, Molecular Devices). The biofilm removal benefit of the enzymes either alone or in combination was expressed as the biofilm reduction percentage (%), which is calculated using the following formula:

Biofilm reduction %=(A1−A2)/A1×100%
A1: A₅₉₅ of the biofilm treated with detergent without enzyme (i.e., control)
A2: A₅₉₅ of the biofilm treated with detergent plus enzyme(s)

The results are summarized in Table 1 below. A greater biofilm reduction % indicates a better biofilm removal benefit.

TABLE 1
Biofilm removal benefit in medical cleaning model detergent MC.

| | | Synergy effect |
|---|---|---|
| | Biofilm | (dual- or tri- combo |
| Conditions | reduction | vs. single enzyme) |
| Control | 0.0% | / |
| 5 ppm Protease 1 | 8.6% | / |
| 5 ppm DNase 3 | 2.4% | / |
| 5 ppm Hexosaminidase 2 | 45.1% | / |
| 5 ppm Protease 1 + 5 ppm DNase 3 | 14.1% | 3% |
| 5 ppm DNase 2 + 5 ppm | 75.7% | 28% |
| Hexosaminidase 2 | | |
| 5 ppm Protease 1 + 5 ppm | 55.2% | 1% |
| Hexosaminidase 2 | | |
| 5 ppm Protease 1 + 5 ppm DNase 3 + | 87.0% | 31% |
| 5 ppm Hexosaminidase 2 | | |

TABLE 2
Biofilm removal benefit in medical cleaning model detergent MC.

| | | Synergy effect |
|---|---|---|
| | Biofilm | (dual- or tri- combo |
| Conditions | reduction | vs. single enzyme) |
| Control | 0.0% | / |
| 5 ppm Protease 2 | 0.1% | / |
| 5 ppm DNase 3 | 7.9% | / |
| 5 ppm Hexosaminidase 2 | 39.6% | / |
| 5 ppm Protease 2 + 5 ppm DNase 3 | 14.7% | 7% |
| 5 ppm DNase 2 + 5 ppm | 63.1% | 16% |
| Hexosaminidase 2 | | |
| 5 ppm Protease 2 + 5 ppm | 40.5% | 1% |
| Hexosaminidase 2 | | |
| 5 ppm Protease 2 + 5 ppm DNase 3 + | 82.4% | 35% |
| 5 ppm Hexosaminidase 2 | | |

TABLE 3
Biofilm removal benefit in medical cleaning model detergent MC.

| | | Synergy effect |
|---|---|---|
| | Biofilm | (dual- or tri- combo |
| Conditions | reduction | vs. single enzyme) |
| Control | 0.0% | / |
| 5 ppm Protease 3 | 2.1% | / |
| 5 ppm DNase 2 | 8.2% | / |
| 5 ppm Hexosaminidase 2 | 37.7% | / |
| 5 ppm Protease 3 + 5 ppm DNase 2 | 16.3% | 6% |
| 5 ppm DNase 2 + 5 ppm | 61.2% | 15% |
| Hexosaminidase 2 | | |
| 5 ppm Protease 3 + 5 ppm | 43.6% | 4% |
| Hexosaminidase 2 | | |
| 5 ppm Protease 3 + 5 ppm DNase 2 + | 69.1% | 21% |
| 5 ppm Hexosaminidase 2 | | |

As can be seen from the above Table 1, Table 2 and Table 3, adding enzymes to the medical cleaning detergent provided a biofilm removal benefit. The combination of two or three enzymes (protease, DNase and/or hexosaminidase) showed a synergy effect on biofilm removal and significantly reduced the biofilm present on the object to be cleaned. This is seen by the biofilm reduction percentage for an enzyme combination being higher than the sum of the biofilm reduction percentages of the individual enzymes. Table 1, Table 2 and Table 3 show that a particularly good effect is obtained by combining a dispersin with a DNase, and especially when a dispersin is combined with both a DNase and a protease.

### Example 2

### Cleaning Test in Medical Cleaning Model Detergent

Staphylococcus aureus 15981 (kind gift from Iñigo Lasa (Valle et al., Mol Microbiol.2003 May; 48 (4): 1075-87) and Staphylococcus epidermidis DSM3270 were used as model microorganisms in the present example. The strains were restreaked on Tryptone Soya Agar (TSA) (pH 7.3) (CM0131; Oxoid Ltd, Basingstoke, UK) and incubated at 37° C. overnight. Single colonies were then inoculated into 10 mL of TSB and incubated for 16 hours at 37° C., 200 rpm. After propagation, the cultures were diluted in fresh TSB+1 wt % glucose (24563; Roquette Freres) to a final ratio of 1:25 (S.aureus: S.epidermidis), and 100 UL aliquots were added to the wells of 96-well polystyrene microplates (167008; Nunc™ MicroWell™ 96-Well, Nunclon Delta-Treated, Flat-Bottom Microplate). Sterile medium was added to control wells. After 18 hours at 37° C. (static incubation), the microplates were aspirated and treated with a model medical cleaning solution (5 g/L model detergent MC in 59H water hardness) with or without enzyme for 1 hour at 30° C. under static conditions. The microplates were then rinsed with a 0.9% NaCl solution and stained with 0.095% crystal violet (SIGMA V5265) for 15 min. Following an additional rinse step, the remaining dye was dissolved using a 30% acetic acid solution. The absorbance was measured at 595 nm. The biofilm removal benefit of the enzymes either alone or in combination was expressed as the biofilm reduction percentage (%), which is calculated as that described in Example 1. The results are displayed in Tables 4-9.

TABLE 4
Synergistic effects between protease and hexosaminidase
in medical cleaning model detergent

| | Enzyme dosage | Biofilm |
|---|---|---|
| Conditions | (μg/ml) | reduction % |
| Model detergent with no enzyme | 0 | 0 |
| Model detergent with protease 4 | 0.5 | 55.3 |
| Model detergent with hexosaminidase 2 | 0.5 | 46.7 |
| Model detergent with hexosaminidase 1 | 0.5 | 52.9 |
| Model detergent with hexosaminidase 4 | 0.5 | 50.3 |
| Model detergent with protease 4 and | 0.25 + 0.25 | 88 |
| hexosaminidase 2 | | |
| Model detergent with protease 4 and | 0.25 + 0.25 | 87.6 |
| hexosaminidase 1 | | |
| Model detergent with protease 4 and | 0.25 + 0.25 | 89.1 |
| hexosaminidase 4 | | |

TABLE 5
Synergistic effects between protease and hexosaminidase
in medical cleaning model detergent

| | Enzyme dosage | Biofilm |
|---|---|---|
| Conditions | (μg/ml) | reduction % |
| Model detergent with no enzyme | 0 | 0 |
| Model detergent with protease 1 | 0.5 | 51.4 |
| Model detergent with hexosaminidase 2 | 0.5 | 52.5 |
| Model detergent with hexosaminidase 1 | 0.5 | 58.1 |
| Model detergent with hexosaminidase 4 | 0.5 | 60.8 |
| Model detergent with protease 1 and | 0.25 + 0.25 | 87.5 |
| hexosaminidase 2 | | |
| Model detergent with protease 1 and | 0.25 + 0.25 | 90.2 |
| hexosaminidase 1 | | |
| Model detergent with protease 1 and | 0.25 + 0.25 | 89.3 |
| hexosaminidase 4 | | |

TABLE 6
Synergistic effects between protease and hexosaminidase
in medical cleaning model detergent

| | Enzyme dosage | Biofilm |
|---|---|---|
| Conditions | (μg/ml) | reduction % |
| Model detergent with no enzyme | 0 | 0 |
| Model detergent with protease 4 | 0.5 | 68.9 |
| Model detergent with hexosaminidase 5 | 0.5 | 51.6 |
| Model detergent with hexosaminidase 3 | 0.5 | 52.3 |
| Model detergent with hexosaminidase 6 | 0.5 | 45.4 |
| Model detergent with protease 4 and | 0.25 + 0.25 | 89.8 |
| hexosaminidase 5 | | |
| Model detergent with protease 4 and | 0.25 + 0.25 | 92.0 |
| hexosaminidase 3 | | |
| Model detergent with protease 4 and | 0.25 + 0.25 | 90.3 |
| hexosaminidase 6 | | |

TABLE 7
Synergistic effects between protease and hexosaminidase
in medical cleaning model detergent

| | Enzyme dosage | Biofilm |
|---|---|---|
| Conditions | (μg/ml) | reduction % |
| Model detergent with no enzyme | 0 | 0 |
| Model detergent with protease 1 | 0.5 | 75.8 |
| Model detergent with hexosaminidase 5 | 0.5 | 45.4 |
| Model detergent with hexosaminidase 3 | 0.5 | 51.7 |
| Model detergent with hexosaminidase 6 | 0.5 | 45.7 |
| Model detergent with protease 1 and | 0.25 + 0.25 | 90.6 |
| hexosaminidase 5 | | |
| Model detergent with protease 1 and | 0.25 + 0.25 | 90.6 |
| hexosaminidase 3 | | |
| Model detergent with protease 1 and | 0.25 + 0.25 | 91.4 |
| hexosaminidase 6 | | |

TABLE 8
Synergistic effects between protease and hexosaminidase
in medical cleaning model detergent

| | Enzyme dosage | Biofilm |
|---|---|---|
| Conditions | (μg/ml) | reduction % |
| Model detergent with no enzyme | 0 | 0 |
| Model detergent with protease 4 | 0.5 | 63.2 |
| Model detergent with hexosaminidase 7 | 2 | 27.2 |
| Model detergent with hexosaminidase 7 | 5 | 24.1 |
| Model detergent with protease 4 and | 0.25 + 1 | 85.5 |
| hexosaminidase 7 | | |

TABLE 9
Synergistic effects between protease and hexosaminidase
in medical cleaning model detergent

| | Enzyme dosage | Biofilm |
|---|---|---|
| Conditions | (μg/ml) | reduction % |
| Model detergent with no enzyme | 0 | 0 |
| Model detergent with protease 1 | 0.5 | 63.7 |
| Model detergent with hexosaminidase 7 | 2 | 28.1 |
| Model detergent with hexosaminidase 7 | 5 | 26.1 |
| Model detergent with protease 1 and | 0.25 + 1 | 87.2 |
| hexosaminidase 7 | | |

The results clearly show that the polypeptides of the invention have cleaning properties in a medical cleaning relevant detergent, i.e. disrupt and/or remove the biofilm or components of the biofilm tested when compared to samples treated with the cleaning solution containing no enzyme. The results also show that an enzyme combination comprising protease and hexosaminidase provides superior cleaning properties in model detergent as compared to the individual enzymes, given that the cleaning performance (biofilm reduction %) of the enzyme combination compared to control detergent without enzyme clearly exceeds the sum of the cleaning performances of the individual enzymes. This clearly suggests that there is a synergetic effect between the two enzymes.

### Example 3

### Cleaning Test in Medical Cleaning Model Detergent

Staphylococcus epidermidis DSM3270 was used as a model microorganism in the present example. The strain was restreaked on Tryptone Soya Agar (TSA) (pH 7.3) (CM0131; Oxoid Ltd, Basingstoke, UK) and incubated at 37° C. overnight. A single colony was then inoculated into 10 mL of TSB and incubated for 16 hours at 37° C., 200 rpm. After propagation, the culture was diluted (1:100) in fresh TSB+1 wt % glucose (24563; Roquette Freres), and 100 UL aliquots were added to the wells of 96-well polystyrene microplates (167008; Nunc™ MicroWell™ 96-Well, Nunclon Delta-Treated, Flat-Bottom Microplate). Sterile medium was added to control wells. After 18 h at 37° C. (static incubation), the microplates were aspirated and treated with a model medical cleaning solution (5 g/L model detergent MC in 5° dH water hardness) with or without enzyme for 1 hour at 30° C. under static conditions. The microplates were then rinsed with a 0.9% NaCl solution and stained with 0.095% crystal violet (SIGMA V5265) for 15 min. Following an additional rinse step, the remaining dye was dissolved using a 30% acetic acid solution. The absorbance was measured at 595 nm. The biofilm removal benefit of the enzymes either alone or in combination was expressed as the biofilm reduction percentage (%), which is calculated as that described in Example 1. The results are displayed in Tables 10-11.

TABLE 10
Synergistic effect between protease and
DNase in medical cleaning model detergent

| | Enzyme | Biofilm |
|---|---|---|
| | dosage | reduction |
| Conditions | (μg/ml) | % |
| Model detergent with no enzyme | 0 | 0 |
| Model detergent with protease 4 | 0.01 | 49.5 |
| Model detergent with DNase 3 | 0.01 | 77.2 |
| Model detergent with DNase 1 | 0.01 | 74.3 |
| Model detergent with protease 4 + DNase 3 | 0.005 + 0.005 | 88 |
| Model detergent with protease 4 + DNase 1 | 0.005 + 0.005 | 84.6 |

TABLE 11
Synergistic effect between protease and
DNase in medical cleaning model detergent

| | Enzyme | Biofilm |
|---|---|---|
| | dosage | reduction |
| Conditions | (μg/ml) | % |
| Model detergent with no enzyme | 0 | 0 |
| Model detergent with protease 1 | 0.01 | 48.8 |
| Model detergent with DNase 3 | 0.01 | 71.8 |
| Model detergent with DNase 1 | 0.01 | 68.8 |
| Model detergent with protease 1 + DNase3 | 0.005 + 0.005 | 84.6 |
| Model detergent with protease 1 + DNase 1 | 0.005 + 0.005 | 79.9 |

The results clearly show that the polypeptides of the invention have cleaning properties in a medical cleaning relevant detergent, i.e. disrupt and/or remove the biofilm or components of the biofilm tested when compared to samples treated with the cleaning solution containing no enzyme. The results also show that an enzyme combination comprising protease and DNase provides superior cleaning properties in model detergent MC as compared to the individual enzymes, given that the cleaning performance (biofilm reduction %) of the enzyme combination clearly exceeds the sum of the performances of the individual enzymes. This clearly suggests that there is a synergetic effect between the two enzymes.

### Example 4

Removal of Pseudomonas aeruginosa (PSA) and PSA Biofilms from Ultrafiltration Membranes

Undesirable microorganisms (e.g. pathogenic bacteria) are not only present in medical devices but may also accumulate in biological manufacturing equipment (e.g., food and beverage manufacturing, and biotech manufacturing such as enzyme fermentation processes), e.g., fermentation and storage tanks, bioreactors, ultrafilters or ultrafiltration membranes, pipelines and other equipment. Such equipment is typically cleaned by cleaning-in-place (CIP) methods, where cleaning is performed without removing or disassembling piping or other equipment. An example of such equipment is the ultrafiltration membranes assembly in a separation module that is often used in an enzyme fermentation process. In between two batches of fermentation, the ultrafiltration membrane assembly is cleaned using a CIP (cleaning-in-place) method. Insufficient cleaning may cause pathogenic bacteria such as PSA to grow and accumulate on the ultrafiltration membrane assembly. On the other hand, bacteria form biofilms to protect themselves from detergents and antibiotics and are thus difficult to remove, which in return facilitates the growth of PSA in ultrafiltration membranes and causes contamination to the fermentation broth. In the present Example 4, the PSA and/or PSA biofilm removal effect of the enzyme combinations of the present invention was evaluated.

### CIP Cleaning Process/Procedure

1. Add enzymes and water to the tank and start the wash procedure to wash the tank and ultrafiltration membrane assembly for 2 hours at 45° C. by the enzyme mixture. The enzymes used in this process and the dosages are:
0.1 g/L of hexosaminidase 2;
0.1 g/L of protease 1; and
0.1 g/L of DNase 3.

2. Rinse the tank and ultrafiltration membrane assembly with water for 10 minutes to remove the released biofilms and PSA.

3. Add Ultrasil 115 and water to the tank (final concentration of Ultrasil 115 is 0.5 wt % in water) and start the wash procedure. Wash the tank and the ultrafiltration membrane assembly for 40 min at 52° C. Ultrasil 115 is a strong alkaline liquid detergent available from Ecolab Inc. In this step, the residual PSA can be further removed or killed by Ultrasil 115.

4. Rinse the tank and ultrafiltration membrane assembly with water for 10 minutes.

5. Add Ultrasil 78 and water to the tank (final concentration of Ultrasil 78 is 0.7 wt % in water). Wash the tank and the ultrafiltration membrane assembly for 40 min at 52° C. Ultrasil 78 is a phosphorus-free acidic liquid detergent available from Ecolab Inc.

6. Rinse the tank and ultrafiltration membrane assembly with water for 10 minutes.

7. Measure the PSA amount in 250 ml of the rinse water of step 6. The results were recorded as “1 wash with enzymes” in below table 12.

Repeat the fermentation process and the CIP wash as described in above steps 1-7, for two more cycles. Then measure the PSA amount in 250 ml of the final rinse water, which is recorded as the “3 wash with enzymes” in below table 12.

TABLE 12
Enzymes combination showed excellent
PSA and biofilms removal effect

| | Amount of PSA |
|---|---|
| Wash without enzymes | 10⁵ cfu/250 ml |
| 1 wash with enzymes (hexosaminidase | 3 × 10² cfu/250 ml |
| 2 + protease 1 + DNase 3) | |
| 3 washes with enzymes (hexosaminidase | <10 cfu/250 ml |
| 2 + protease 1 + DNase 3) | |

It is clear from Table 12 that by using the enzymes of the present invention in the CIP cleaning process, the removal of PSA can be significantly improved. Using enzymes in the CIP process after each batch of fermentation can further improve PAS removal and maintain PSA at an extremely low level (<10 cfu/250 ml after e.g. 3 washes), a level that is desirable especially in a food-related production process.

### Example 5

### Biofilm Removal Test in Medical Cleaning Model Detergent MC

In Example 5, different enzymes/dosages were evaluated. The experimental procedure is the same as that described in Example 1. The results are summarized in below tables 13-15.

TABLE 13
Biofilm removal benefit in medical cleaning model detergent MC.

| | | Synergy effect |
|---|---|---|
| | Biofilm | (dual- or tri- combo |
| Conditions | reduction | vs. single enzyme) |
| Control | 0.0% | / |
| 5 ppm Protease 1 | 4.3% | / |
| 5 ppm DNase 4 | 4.8% | / |
| 5 ppm Hexosaminidase 2 | 31.0% | / |
| 5 ppm Protease 1 + 5 ppm DNase 4 | 17.2% | 8.1% |
| 5 ppm DNase 4 + 5 ppm | 70.2% | 34.4% |
| Hexosaminidase 2 | | |
| 5 ppm Protease 1 + 5 ppm | 61.3% | 26.0% |
| Hexosaminidase 2 | | |
| 5 ppm Protease 1 + 5 ppm DNase 4 + | 85.7% | 45.6% |
| 5 ppm Hexosaminidase 2 | | |

TABLE 14
Biofilm removal benefit in medical cleaning model detergent MC.

| | | Synergy effect |
|---|---|---|
| | Biofilm | (dual- or tri- combo |
| Conditions | reduction | vs. single enzyme) |
| Control | 0% | / |
| 5 ppm Protease 1 | 12.6% | / |
| 5 ppm DNase 5 | 2.3% | / |
| 5 ppm Hexosaminidase 2 | 49.3% | / |
| 5 ppm Protease 1 + 5 ppm DNase 5 | 23.8% | 8.9% |
| 5 ppm DNase 5 + 5 ppm | 77.8% | 26.1% |
| Hexosaminidase 2 | | |
| 5 ppm Protease 1 + 5 ppm | 62.4% | 0.4% |
| Hexosaminidase 2 | | |
| 5 ppm Protease 1 + 5 ppm DNase 5 + | 88.6% | 24.4% |
| 5 ppm Hexosaminidase 2 | | |

TABLE 15
Biofilm removal benefit in medical cleaning model detergent MC.

| | | Synergy effect |
|---|---|---|
| | Biofilm | (dual- or tri- combo |
| Conditions | reduction | vs. single enzyme) |
| Control | 0.0% | / |
| 18.75 ppm Protease 1 | 4.3% | / |
| 2 ppm DNase 6 | 0.3% | / |
| 2 ppm Hexosaminidase 2 | 12.2% | / |
| 18.75 ppm Protease 1 + 2 ppm | 7.5% | 2.9% |
| DNase 6 | | |
| 2 ppm DNase 6 + 2 ppm | 31.4% | 18.9% |
| Hexosaminidase 2 | | |
| 18.75 ppm Protease 1 + 2 ppm | 43.5% | 27.0% |
| Hexosaminidase 2 | | |
| 18.75 ppm Protease 1 + 2 ppm | 71.2% | 54.4% |
| DNase 6 + 2 ppm Hexosaminidase 2 | | |

1. A method of cleaning a medical device, comprising the steps of:
(a) contacting the medical device with a wash liquor comprising two or more enzymes selected from the group consisting of a protease, an enzyme having DNase activity and an enzyme having hexosaminidase activity; and optionally
(b) rinsing the medical device.

2. The method according to claim 1, wherein the medical device is or comprises an endoscope such as a cystoscope, a nephroscope, a bronchoscope, a laryngoscope, an otoscope, an arthroscope, a laparoscope, and a gastrointestinal endoscope; a surgical instrument such as a scalpel, a hemostat, a forceps, scissors, a retractor, a tracheotome and a clamp; and dental instruments e.g. scalers, curettes, serrated cotton pliers, dental mirrors.

3. The method according to claim 1, wherein the medical device is soiled with biofilm, wherein the biofilm is produced by or partly produced by Escherichia coli, Klebsiella pneumoniae, Salmonella, Mycobacterium, Enterococcus faecalis, Enterobacter cloacae, Proteus mirabilis, Serratia marcescens, Staphylococcus aureus such as Staphylococcus aureus ATCC 6538, Streptococcus (e.g., S. pyogenes, S. agalacticae or S. pneumoniae), Haemophilus influenzae, Pseudomonas aeruginosa, Clostridium perfringens, Chlamydia trachomatis, Candida albicans, and/or Bacillus anthracis.

4. The method according to claim 1, wherein the enzyme having DNase activity is a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 1, 2, 3 or 4.

5. The method according to claim 1, wherein the enzyme having DNase activity is a variant of SEQ ID NO: 2, comprising one or more alterations selected from the group consisting of S26 D32E,Q, V35I, K36C,H, G37R, F43W, D46G, A55I, N68D, S69V, A76I, K82S, T, P84D, T, K86E,G,L,N,Q,T, V,Y, A91R, L92E, K95I, P97E,N, A101E, Q102E, K105N,G,Q,T,D, A111P, F112Y,W, S115T, V127T, L129K, N133Q, G137R, V138C, N140H, G141Q,R, S144E, N146A, K147N,E, V148I, A149D,E,F, Q150D,E, P153D,V, S154E, K155E,F,L,S,T, Q157D,E, Q158D, T159Q, K160D, G161R, T170Q, A172D,E,H,R, G181N, K185*, V187N, Y, N191*, K192A,I, D197K,S, G199Q, Q208V, E211Y,T,P, N213S, N214D, N217A and Y218D,E, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

6. The method according to claim 1, wherein the enzyme having hexosaminidase activity is a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10, 11, 19, 20, 21 or 22.

7. The method according to claim 1, wherein the enzyme having hexosaminidase activity is a variant of polypeptide of SEQ ID NO: 10, comprising one or more alterations at positions 3, 15, 49, 59, 163, 186, 225, 227, 232, 235, 252, 260, 272, 279, 281, 308, 309 and 312 of SEQ ID NO: 10, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 10.

8. The method according to claim 1, wherein the protease has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 5, 6, 7, 8, 9 or 29.

9. The method according to claim 8, wherein the protease is a variant of the polypeptide of SEQ ID NO: 6 comprising an alteration at one or more positions corresponding to positions 3, 4, 9, 15, 22, 43, 68, 76, 87, 99, 101, 103, 104, 118, 128, 160, 167, 170, 184, 194, 199, 205, 206, 209, 217, 218, 222, 245, 259, 261 and 262, wherein position numbers correspond to the positions of SEQ ID NO: 5, wherein each alteration is independently a substitution, deletion or insertion, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 6.

10. The method according to claim 1, wherein the protease is:
(a) a variant of the polypeptide of SEQ ID NO: 6, comprising one or more substitutions selected from the group consisting of: S3T, V4I, S9E, S9R, A15T, T22A, N43R, V68A, N76D, S87N, S99D, S99G, S99A, S99SE, S101E, S101N, S101R, S103A, V104I, G118M, S128Q, G160S, Y167A, R170S, N184E, A194P, V199M, V205I, Q206L, Y209W, L217D, L217Q, N218D, M222S, Q245R, S259D, N261W and L262E, e.g., comprising substitutions of N76D+Q206L+Y209W or Y209W+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, at least 99%, but less than 100% sequence identity to SEQ ID NO: 6;
(b) a variant of the polypeptide of SEQ ID NO: 6, comprising the substitution S87N, wherein the variant has protease activity and wherein the position corresponds to the position of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(c) a variant of the polypeptide of SEQ ID NO: 6, comprising the substitutions Y167A+R170S+A194P, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(d) a variant of the polypeptide of SEQ ID NO: 6, comprising the substitutions S9E+N43R+N76D+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(e) a variant of the polypeptide of SEQ ID NO: 6, comprising the substitutions S3T+N43R+N76D+S87N+G118M+S128Q+N184E+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(f) a variant of the polypeptide of SEQ ID NO: 6, comprising the substitutions T22A+N43R+S87N+V205L+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(g) a variant of the polypeptide of SEQ ID NO: 8, comprising the substitutions A68S+T77N+T78I+G127S+A128P+G165Q+N184Q+A202V+N217S+S258P, wherein position numbers correspond to the positions of SEQ ID NO: 8, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO:8;
(h) a variant comprising a substitution at one or more positions corresponding to positions 171, 173, 175, 179 or 180 of SEQ ID NO: 1 of WO2004/067737, wherein the variant has protease activity and has a sequence identity of at least 80%, at least 85%, at least 90%, at least 91%, at least 92%, at least 93%, at least 94%, at least 95%, at least 96%, at least 97%, at least 98%, or at least 99%, but less than 100% to SEQ ID NO: 1 of WO2004/067737;
(i) a protease variant comprising one or more substitutions compared to a parent protease, selected from the group consisting of X3V, X9 [E,R], X22 [R,A], X43R, X61 [E,D], X62 [E,D], X76 [D], X87N, X101 [E,G,D,N,M], X103A, X104I, X118 [V,R,M], X120V, X128 [A,L,S,Q], X129Q, X130A, X160D, X184 [E,D], X185 [E,D], 188 [E,D], X191N, X194P, X205I, X206L, X209W, X216V, X217 [Q,D,E], X218 [D,E,S], X232V, X245R, X248D, X256 [E,D], X259 [E,D], X261 [E,D,W] and X262 [E,D], wherein position numbers correspond to the positions of BPN′(SEQ ID NO: 5), wherein “X” represents any amino acid residue present in the specified position in the parent protease, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or SEQ ID NO: 6; and
(j) a protease variant comprising any of the following substitution sets compared to a parent protease, wherein the parent protease has the amino acid sequence of SEQ ID NO: 5 or SEQ ID NO: 6 or has at least 80% sequence identity to SEQ ID NO: 5 or SEQ ID NO: 6, wherein position numbers correspond to the positions of BPN′ (SEQ ID NO: 5), wherein “X” represents any amino acid residue present in the specified position in the parent protease, and wherein the substitution set is selected from the group consisting of:
i. X9R+X15T+X68A+X218D+X245R,
ii. X9R+X15T+X68A+X245R,
iii. X61E+X194P+X205I+X261D,
iv. X61D+X205I+X245R,
v. X61E+X194P+X205I+X261D,
vi. X87N+X118V+X128L+X129Q+X130A,
vii. X87N+X101M+X118V+X128L+X129Q+X130A,
viii. X76D+X87R+X118R+X128L+X129Q+X130A,
ix. X22A+X62D+X101G+X188D+X232V+X245R,
X. X103A+X104I,
xi. X22R+X101G+X232V+X245R,
xii. X103A+X104I+X156D,
xiii. X103A+X104I+X261E,
xiv. X62D+X245R,
xv. X101N+X128A+X217Q,
xvi. X101E+X217Q,
xvii. X101E+X217D,
xviii. X9E+X43R+X262E,
xix. X76D+X43R+X209W,
xx. X205I+X206L+X209W,
xxi. X185E+X188E+X205I,
xxii. X256D+X261W+X262E,
xxiii. X191N+X209W,
xxiv. X261E+X262E,
xxv. X261E+X262D, and
xxvi. X167A+X170S+X194P,
wherein the protease variant has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or 6.

11. (canceled)

12. A composition for cleaning a medical device, comprising a surfactant and two or more enzymes selected from the group consisting of a protease, an enzyme having DNase activity and an enzyme having hexosaminidase activity.

13. The composition according to claim 12, wherein the enzyme having DNase activity is a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 1, 2, 3 or 4.

14. The composition according to claim 12, wherein the enzyme having hexosaminidase activity is a polypeptide having at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10, 11, 19, 20, 21 or 22.

15. The composition according to claim 12, wherein the protease has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 5, 6, 7, 8, 9 or 29.

16. The composition according to claim 12, wherein the enzyme having DNase activity is a variant of SEQ ID NO: 2, comprising one or more alterations selected from the group consisting of S26 D32E,Q, V35I, K36C,H, G37R, F43W, D46G, A55I, N68D, S69V, A76I, K82S,T, P84D,T, K86E,G,L,N,Q,T,V,Y, A91R, L92E, K95I, P97E,N, A101E, Q102E, K105N,G,Q,T,D, A111P, F112Y,W, S115T, V127T, L129K, N133Q, G137R, V138C, N140H, G141Q,R, S144E, N146A, K147N,E, V148I, A149D,E,F, Q150D,E, P153D,V, S154E, K155E,F,L,S,T, Q157D,E, Q158D, T159Q, K160D, G161R, T170Q, A172D,E,H,R, G181N, K185*, V187N,Y, N191*, K192A,I, D197K,S, G199Q, Q208V, E211Y,T,P, N213S, N214D, N217A and Y218D,E, wherein position numbers correspond to the positions of SEQ ID NO: 2, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2 or 3.

17. The composition according to claim 12, wherein the enzyme having hexosaminidase activity is a variant of polypeptide of SEQ ID NO:10, comprising one or more alterations at positions 3, 15, 49, 59, 163, 186, 225, 227, 232, 235, 252, 260, 272, 279, 281, 308, 309 and 312 of SEQ ID NO: 10, wherein the variant has at least 80%, such as at least 85%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 10.

18. The composition according to according to claim 12, wherein the protease is a variant of the polypeptide of SEQ ID NO: 6 comprising an alteration at one or more positions corresponding to positions 3, 4, 9, 15, 22, 43, 68, 76, 87, 99, 101, 103, 104, 118, 128, 160, 167, 170, 184, 194, 199, 205, 206, 209, 217, 218, 222, 245, 259, 261 and 262, wherein position numbers correspond to the positions of SEQ ID NO: 5, wherein each alteration is independently a substitution, deletion or insertion, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 6.

19. The composition according to claim 12, wherein the protease is:
(a) a variant of the polypeptide of SEQ ID NO: 6, comprising one or more substitutions selected from the group consisting of: S3T, V4I, S9E, S9R, A15T, T22A, N43R, V68A, N76D, S87N, S99D, S99G, S99A, S99SE, S101E, S101N, S101R, S103A, V104I, G118M, S128Q, G160S, Y167A, R170S, N184E, A194P, V199M, V205I, Q206L, Y209W, L217D, L217Q, N218D, M222S, Q245R, S259D, N261W and L262E, e.g., comprising substitutions of N76D+Q206L+Y209W or Y209W+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, at least 99%, but less than 100% sequence identity to SEQ ID NO: 6;
(b) a variant of the polypeptide of SEQ ID NO: 6, comprising the substitution S87N, wherein the variant has protease activity and wherein the position corresponds to the position of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(c) a variant of the polypeptide of SEQ ID NO: 6, comprising the substitutions Y167A+R170S+A194P, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(d) a variant of the polypeptide of SEQ ID NO: 6, comprising the substitutions S9E+N43R+N76D+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(e) a variant of the polypeptide of SEQ ID NO: 6, comprising the substitutions S3T+N43R+N76D+S87N+G118M+S128Q+N184E+V205I+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(f) a variant of the polypeptide of SEQ ID NO: 6, comprising the substitutions T22A+N43R+S87N+V205L+Q206L+Y209W+S259D+N261W+L262E, wherein position numbers correspond to the positions of SEQ ID NO: 5, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 6;
(g) a variant of the polypeptide of SEQ ID NO: 8, comprising the substitutions A68S+T77N+T78I+G127S+A128P+G165Q+N184Q+A202V+N217S+S258P, wherein position numbers correspond to the positions of SEQ ID NO: 8, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO:8;
(h) a variant comprising a substitution at one or more positions corresponding to positions 171, 173, 175, 179 or 180 of SEQ ID NO: 1 of WO2004/067737, wherein the variant has protease activity and has a sequence identity of at least 80%, at least 85%, at least 90%, at least 91%, at least 92%, at least 93%, at least 94%, at least 95%, at least 96%, at least 97%, at least 98%, or at least 99%, but less than 100% to SEQ ID NO: 1 of WO2004/067737;
(i) a protease variant comprising one or more substitutions compared to a parent protease, selected from the group consisting of X3V, X9 [E, R], X22 [R,A], X43R, X61 [E,D], X62 [E,D], X76 [D], X87N, X101 [E,G,D,N,M], X103A, X104I, X118 [V,R,M], X120V, X128 [A,L,S,Q], X129Q, X130A, X160D, X184 [E,D], X185 [E,D], 188 [E,D], X191N, X194P, X205I, X206L, X209W, X216V, X217 [Q,D,E], X218 [D,E,S], X232V, X245R, X248D, X256 [E,D], X259 [E,D], X261 [E,D,W] and X262 [E, D], wherein position numbers correspond to the positions of BPN′ (SEQ ID NO: 5), wherein “X” represents any amino acid residue present in the specified position in the parent protease, and wherein the variant has protease activity and has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or SEQ ID NO: 6; and
(j) a protease variant comprising any of the following substitution sets compared to a parent protease, wherein the parent protease has the amino acid sequence of SEQ ID NO: 5 or SEQ ID NO: 6 or has at least 80% sequence identity to SEQ ID NO: 5 or SEQ ID NO: 6, wherein position numbers correspond to the positions of BPN′ (SEQ ID NO: 5), wherein “X” represents any amino acid residue present in the specified position in the parent protease, and wherein the substitution set is selected from the group consisting of:
i. X9R+X15T+X68A+X218D+X245R,
ii. X9R+X15T+X68A+X245R,
iii. X61E+X194P+X205I+X261D,
iv. X61D+X205I+X245R,
v. X61E+X194P+X205I+X261D,
vi. X87N+X118V+X128L+X129Q+X130A,
vii. X87N+X101M+X118V+X128L+X129Q+X130A,
viii. X76D+X87R+X118R+X128L+X129Q+X130A,
ix. X22A+X62D+X101G+X188D+X232V+X245R,
X. X103A+X104I,
xi. X22R+X101G+X232V+X245R,
xii. X103A+X104I+X156D,
xiii. X103A+X104I+X261E,
xiv. X62D+X245R,
xv. X101N+X128A+X217Q,
xvi. X101E+X217Q,
xvii. X101E+X217D,
xviii. X9E+X43R+X262E,
xix. X76D+X43R+X209W,
xx. X205I+X206L+X209W,
xxi. X185E+X188E+X205I,
xxii. X256D+X261W+X262E,
xxiii. X191N+X209W,
xxiv. X261E+X262E,
xxv. X261E+X262D, and
xxvi. X167A+X170S+X194P,
wherein the protease variant has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 5 or 6.
